(19) United States
(12) Patent Application Publication
(10) Pub. No.: US 2003/0235811 A1
(43) Pub. Date: Dec. 25, 2003

(54) Crystallized mammalian carboxylesterase polypeptide and screening methods employing same

(75) Inventors: Matthew R. Redinbo, Carrboro, NC (US); Sompop Bencharit, Chapel Hill, NC (US); Christopher L. Morton, Memphis, TN (US); Philip M. Potter, Memphis, TN (US)

Correspondence Address: JENKINS & WILSON, PA, 3100 TOWER BLVD, SUITE 1400, DURHAM, NC, 27707, US

(73) Assignee: The University of North Carolina at Chapel Hill

(21) Appl. No.: 10/267,756

(22) Filed: Oct. 9, 2002

Related U.S. Application Data
(60) Provisional application No. 60/374,513, filed on Apr. 22, 2002.

(51) Int. Cl. C12Q 1/00; G06F 19/00; G01N 33/48; G01N 33/50; C12N 9/16

(52) U.S. Cl. 435/4; 435/196; 702/19

(57) ABSTRACT

Solved three-dimensional crystal structures of mammalian carboxylesterases (CEs) are disclosed. A solved three-dimensional crystal structure of a rabbit CE polypeptide co-crystallized with 4PP is disclosed. Solved three-dimensional structures of a human CE polypeptide co-crystallized with tacrine and a human CE polypeptide co-crystallized with homatropine are disclosed. The disclosed structures can be employed in the design of CE modulators. Methods of designing modulators of the biological activity of rabbit CE, human CE and other CE polypeptides, are also disclosed.

### CROSS REFERENCE TO RELATED APPLICATIONS

This application is based on and claims priority to U.S. Provisional Patent Application Serial No. 60/374,513, filed Apr. 22, 2002, the entire contents of which are herein incorporated by reference.

### TECHNICAL FIELD

The present invention relates generally to the structure of mammalian carboxylesterase (CE) polypeptide, and more particularly to the crystalline structure of mammalian carboxylesterase (CE) polypeptides. The invention further relates to methods by which modulators and ligands of CE can be identified and designed.

Abbreviations

| | |
|---|---|
| 4PP | 4-piperidino-piperidine |
| AcChE | acetylcholinesterase |
| ACE | angiotension-converting enzyme |
| AD | Alzheimer's disease |
| ADP | adenosine diphosphate |
| ATP | adenosine triphosphate |
| BSA | bovine serum albumin |
| βME | β-mercaptoethanol |
| CE | carboxylesterase |
| CPT-11 | irinotecan |
| DMSO | dimethyl sulfoxide |
| DNA | deoxyribonucleic acid |
| DTT | dithiothreitol |
| EDTA | ethylenediaminetetraacetic acid |
| FAEE | fatty acid ethyl esters |
| hCE1 | human carboxylesterase I |
| hiCE | human intestinal CE |
| HEPES | N-2-Hydroxyethylpiperazine-N′-2-ethanesulfonic |
| | acid |
| kDa | kilodalton(s) |
| MAD | multiwavelength anomalous diffraction |
| MAN | mannose |
| NAG | N-acetylglucosamine |
| NDP | nucleotide diphosphate |
| nt | nucleotide |
| NTP | nucleotide triphosphate |
| PAGE | polyacrylamide gel electrophoresis |
| PCR | polymerase chain reaction |
| pI | isoelectric point |
| rhncCEH | rat hepatic neutral cytosolic cholseteryl ester |
| | hydrolase |
| rLCE | rat lung carboxylesterase |
| rLCE | rat lung CE |
| rhncCEH | rat hepatic neutral cytosolic cholesteryl ester |
| | hydrolase |
| RMSD | root-mean-square deviation |
| SDS | sodium dodecyl sulfate |
| SDS-PAGE | sodium dodecyl sulfate polyacrylamide gel |
| | electrophoresis |
| SIRAS | single isomorphous replacement anomalous |
| | scattering |
| SN-38 | a potent topoisomerase I-specific poison |
| SSRL | Stanford Synchrotron Radiation Laboratory |
| tAcChE | Torpedo cailfornica acetylcholinesterase |
| WT | wildtype |

Amino Acid Abbreviations

| Single-Letter Code | Three-Letter Code | Name |
|---|---|---|
| A | Ala | Alanine |
| V | Val | Valine |
| L | Leu | Leucine |
| I | Ile | Isoleucine |
| P | Pro | Proline |
| F | Phe | Phenylalanine |
| W | Trp | Tryptophan |
| M | Met | Methionine |
| G | Gly | Glycine |
| S | Ser | Serine |
| T | Thr | Threonine |
| C | Cys | Cysteine |
| Y | Tyr | Tyrosine |
| N | Asn | Asparagine |
| Q | Gln | Glutamine |
| D | Asp | Aspartic Acid |
| E | Glu | Glutamic Acid |
| K | Lys | Lysine |
| R | Arg | Arginine |
| H | His | Histidine |

Functionally Equivalent Codons

| Amino Acid | | | Codons |
|---|---|---|---|
| Alanine | Ala | A | GCA GCC GCG GGU |
| Cysteine | Gys | C | UGG UGU |
| Aspartic Acid | Asp | D | GAG GAU |
| Glumatic acid | Glu | E | GAA GAG |
| Phenylalanine | Phe | F | UUG UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Methionine | Met | M | AUG |
| Asparagine | Asn | N | AAC AAU |
| Proline | Pro | P | CCA CCC CCG CCU |
| Glutamine | Gln | Q | CAA CAG |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |
| Leucine | Leu | L | UUA UUG CUA CUC |
| | | | CUG CUU |
| Arginine | Arg | R | AGA AGG CGA CGC |
| | | | CGG CGU |
| Serine | Ser | S | ACG AGU UCA UCC |
| | | | UCG UCU |

### BACKGROUND ART

Unraveling the structural basis of how carboxylesterases (CE) recognize an array of different endogenous and exogenous compounds, including both small and large ligands, is central to understanding how compounds are cleared from the body. Such knowledge will improve our understanding of the metabolism of xenobiotics and may aid in the design of therapeutics for the treatment of cancer and other conditions.

Human carboxylesterase 1 (hCE1) plays central roles in several key biological processes. This enzyme catalyzes the hydrolysis of esters, thioesters and amide bonds in a wide variety of chemically distinct drugs, xenobiotics and endogenous compounds. Its primary role appears to be in the promiscuous metabolism and detoxification of xenobiotics that pose a potential threat to our survival. However, this enzyme also plays a role in the activation of prodrugs in humans, as well as in cholesterol trafficking, lung function, sex hormone maturation, and in the entrance of the malaria parasite to human liver. hCE1 (Entrez-Protein nucleotide accession code NM_{—}001266, accessible via http://www.ncbi.nlm.nih.gov/; Mori et al., (1999) FEBS Lett. 458 (1): 17-22) comprises 566 amino acids with two disulfide linkages and one site of asparagine-linked glycosylation. It is related to an hCE1 isoform (Entrez-Protein protein accession code AAA16036, accessible via http://www.ncbi.nlm.nih.gov/; Kroetz et al., (1993) Biochem. 32 (43): 11606-11617), which comprises 568 amino acids in length and contains two amino acids changes: an alanine is inserted at position 17, and a glutamine is inserted at position 362.

Polypeptides, including mammalian CEs, have a three-dimensional structure determined by the primary amino acid sequence and the environment surrounding the polypeptide. This three-dimensional structure establishes the polypeptide's activity, stability, binding affinity, binding specificity, and other biochemical attributes. Thus, knowledge of a protein's three-dimensional structure can provide much guidance in designing agents that mimic, inhibit, or improve the protein's biological activity in soluble or membrane bound forms.

The three-dimensional structure of a polypeptide can be determined in a number of ways. Many of the most precise methods employ X-ray crystallography (see, e.g., Van Holde, (1971) Physical Biochemistry, Prentice-Hall, New Jersey, pp. 221-39). This technique relies on the ability of crystalline lattices to diffract X-rays or other forms of radiation. Diffraction experiments suitable for determining the three-dimensional structure of macromolecules typically require high-quality crystals. Unfortunately, such crystals have been unavailable for a mammalian CE, as well as many other proteins of interest. Thus, high-quality diffracting crystals of a mammalian CE would greatly assist in the elucidation of CE's three-dimensional structure, and would provide insight into the ligand binding properties of CE.

Clearly, a solved crystal structure of a mammalian CE would be useful in the design of modulators of activity mediated by all CE isoforms. Evaluation of the available sequence data indicates that CE shows structural homology with the three-dimensional structure of other proteins (see, e.g., FIG. 4). Thus, the three-dimensional structure of CE could also be employed to study and design modulators of other proteins.

A solved mammalian CE-ligand crystal structure would provide structural details and insights necessary to design a modulator of CE that maximizes desirable characteristics for any modulator, e.g. potency and specificity. By exploiting the structural details obtained from a CE-ligand crystal structure, it would be possible to design a CE modulator that, despite CE's similarity with other proteins, exploits the unique structural features of CE. A CE modulator developed using structure-assisted design would take advantage of heretofore unknown CE structural considerations and thus be more effective than a modulator developed using homology-based design. Potential or existent homology models cannot provide the necessary degree of specificity. A CE modulator designed using the structural coordinates of a crystalline form of a mammalian CE would also provide a starting point for the development of modulators of other structurally similar proteins.

What is needed, therefore, is a crystallized form of a mammalian CE polypeptide, and in another embodiment, a mammalian CE polypeptide in complex with a ligand. Acquisition of crystals of a mammalian CE polypeptide will permit the three dimensional structure of the mammalian CE to be determined. Knowledge of this three dimensional structure will facilitate the design of modulators of CE activity. Such modulators can lead to therapeutic compositions to treat a wide range of conditions, including cancer, conditions associated with toxic endogenous compounds and xenobiotics, activation of prodrugs, cholesteryl ester formation and hydrolysis, sex hormone maturation, lung surfactant generation, treatment of Alzheimer's disease, fatty acid ethyl ester formation associated with alcohol abuse and malaria invasion into human liver, cholesterol and fatty acid metabolism, narcotic abuse and overdose treatments to name just a few applications.

### SUMMARY OF THE INVENTION

A substantially pure mammalian CE polypeptide in crystalline form is presented. In one embodiment, the mammalian CE polypeptide is a rabbit CE polypeptide. In another embodiment, the polypeptide is in rhombohedral crystalline form with lattice constants of a=110.23 Å, b=110.23 Å, c=282.52 Å, α=90°, β=90°, γ=120°, and a space group of R32. In yet another embodiment, the crystalline form contains one CE polypeptide in the asymmetric unit. The crystalline form can optionally comprise one or more atoms having atomic weight of 40 grams/mol or greater, e.g. a heavy atom derivative. In an additional embodiment, the coordinates of Table 2 further characterize the CE.

In another embodiment, the substantially pure mammalian CE polypeptide is a human CE polypeptide. In yet another embodiment, the polypeptide is in monoclinic crystalline form is has lattice constants selected from the group consisting of a=90.0 Å, b=117.0 Å, c=176.0 Å, α=90°, β=95.7°, γ=90°; and a=55.4 Å, b=178.8 Å, c=199.6 Å, α=90°, β=90.2°, γ=90°, and a space group of P2₁. In a further embodiment, the crystalline form contains six CE polypeptides in the asymmetric unit. The crystalline form can optionally comprise one or more atoms having atomic weight of 40 grams/mol or greater, e.g. a heavy atom derivative. In an additional embodiment, the coordinates corresponding to Tables 6 and 7 further characterize the CE.

A method for determining the three-dimensional structure of a crystallized mammalian CE polypeptide to a resolution of about 2.8 Å or better is disclosed. In one embodiment, the method comprises: (a) crystallizing a mammalian CE polypeptide by sitting drop vapor diffusion wherein CE polypeptide is mixed with an equal volume of reservoir comprising PEG3350, 0.1M Li₂SO₄, 0.1M citrate, pH 5.5 and 5% glycerol; and (b) analyzing the mammalian CE polypeptide by X-ray diffraction to determine the three-dimensional structure.

A method of generating a crystallized mammalian CE ligand binding domain polypeptide is disclosed. In one embodiment, the method comprises: (a) incubating a solution comprising a mammalian CE ligand binding domain with an equal volume of reservoir; and (b) crystallizing the mammalian CE polypeptide using the sitting drop method, whereby a crystallized mammalian CE polypeptide is generated.

A method of designing a modulator of a mammalian CE polypeptide is disclosed. In one embodiment, the method comprises: (a) designing a potential modulator of a mammalian CE polypeptide that will form bonds with amino acids in a ligand binding site based upon a crystalline structure of a mammalian CE polypeptide; (b) synthesizing the modulator; and (c) determining whether the potential modulator modulates the activity of the mammalian CE polypeptide, whereby a modulator of a mammalian CE polypeptide is designed.

A method of designing a modulator that selectively modulates the activity of a human CE polypeptide to the exclusion of other CE polypeptides is disclosed. In one embodiment, the method comprises: (a) obtaining a crystalline form of the mammalian CE polypeptide; (b) evaluating the three-dimensional structure of the crystallized mammalian CE polypeptide; and (c) synthesizing a potential modulator based upon the three dimensional crystal structure of the crystallized mammalian CE ligand binding domain polypeptide, whereby a modulator that selectively modulates the activity of a human CE polypeptide to the exclusion of other CE polypeptides is designed.

The foregoing method optionally further comprises contacting a human CE polypeptide with the potential modulator; and assaying the human CE polypeptide for binding of the potential modulator, for a change in activity of the human CE polypeptide, or both.

A method for identifying a CE modulator is disclosed. In one embodiment, the method comprises: (a) providing atomic coordinates of a mammalian CE polypeptide to a computerized modeling system; and (b) modeling a ligand that fits spatially into a binding cavity or on the surface of the mammalian CE ligand binding domain, whereby a CE modulator is identified. The method can optionally further comprise identifying in an assay for CE-mediated activity a modeled ligand that increases or decreases the activity of the CE.

A method of identifying a CE modulator that selectively modulates the activity of a CE polypeptide compared to other polypeptides is disclosed. In one embodiment, the method comprises: (a) providing atomic coordinates of a mammalian CE polypeptide to a computerized modeling system; and (b) modeling a ligand that fits spatially into a binding cavity or on the surface of a mammalian CE polypeptide and that interacts with conformationally constrained residues of a CE that are conserved among CE orthologs and isoforms, whereby a CE modulator is identified. The method can optionally further comprise identifying in a biological assay for CE-mediated activity a modeled ligand that selectively binds to the CE polypeptide and increases or decreases the activity of the CE.

A method of identifying a modulator of a CE polypeptide is disclosed. In one embodiment, the method comprises: (a) selecting a candidate CE ligand; (b) determining which amino acid or amino acids of a CE polypeptide interact with the ligand using a three-dimensional model of a crystallized protein comprising a mammalian CE; (c) identifying in a biological assay for CE activity a degree to which the ligand modulates the activity of the CE polypeptide; (d) selecting a chemical modification of the ligand wherein the interaction between the amino acids of the CE polypeptide and the ligand is predicted to be modulated by the chemical modification; (e) performing the chemical modification on the ligand to form a modified ligand; (f) contacting the modified ligand with the CE polypeptide; (g) identifying in a biological assay for CE activity a degree to which the modified ligand modulates the biological activity of the CE polypeptide; and (h) comparing the biological activity of the CE polypeptide in the presence of modified ligand with the biological activity of the CE polypeptide in the presence of the unmodified ligand, whereby a modulator of a CE polypeptide is designed. The method can employ human CE polypeptide as the mammalian CE polypeptide, or rabbit CE as the mammalian CE polypeptide. The method can optionally further comprise repeating steps (a) through (f), if the biological activity of the CE polypeptide in the presence of the modified ligand varies from the biological activity of the CE polypeptide in the presence of the unmodified ligand.

An assay method for identifying a compound that inhibits binding of a ligand to a CE polypeptide is disclosed. In one embodiment, the method comprises: (a) designing a test inhibitor compound capable of modulating CE activity based on the atomic coordinates of a mammalian CE polypeptide; (b) synthesizing the test inhibitor compound; (c) incubating a CE polypeptide with a ligand in the presence of a test inhibitor compound; and (d) determining an amount of ligand that is bound to the CE polypeptide, wherein decreased binding of ligand to the CE protein in the presence of the test inhibitor compound is indicative of inhibition, whereby a compound that inhibits binding of a ligand to a CE polypeptide is identified.

A method of modeling a three-dimensional structure of a target CE in complex with a ligand from a template comprising the X-ray structure of a mammalian CE complex with a ligand is disclosed. In one embodiment, the method comprises: (a) selecting an X-ray structure of a target CE built by homology modeling as a starting model for the target CE; (b) manipulating the starting model for the target CE as a rigid body to superimpose its backbone atoms onto corresponding backbone atoms of a three-dimensional template structure (e.g. characterized by the coordinates of Tables 2, 6 or 7) comprising a mammalian CE in complex with a ligand to form a manipulated model; (c) making a copy of the ligand from the template structure to form a model of a ligand bound to a template mammalian CE; (d) merging the model of the ligand into the manipulated model to form a modified model; (e) removing one or more amino acids from the modified model; and (f) optimizing side-chain conformations using varying distance constraints, whereby a three-dimensional structure of a target CE in complex with a ligand is modeled from a template comprising the X-ray structure of a mammalian CE in complex with a ligand.

A method of screening a plurality of compounds for a modulator of a CE polypeptide is disclosed. In one embodiment, the method comprises: (a) providing a library of test samples bound to a substrate; (b) contacting a crystalline form comprising a mammalian CE in complex with a ligand with each test sample; (c) detecting an interaction between a test sample and the crystalline mammalian CE polypeptide in complex with a ligand; (d) identifying a test sample that interacts with the crystalline mammalian CE polypeptide in complex with a ligand; and (e) isolating a test sample that interacts with the crystalline mammalian CE polypeptide in complex with a ligand, whereby a plurality of compounds is screened for a modulator of a CE ligand binding domain polypeptide.

In each of the foregoing embodiments of a method of the present invention, a mammalian CE can be, for example, a rabbit CE or a human CE and can comprise an amino acid sequence as set forth in SEQ ID NO: 2 or 4. Furthermore, in each of the methods of the present invention, the crystalline form can be such that the three-dimensional structure of the crystallized mammalian CE polypeptide is determined to a resolution of about 2.8 Å or better. In addition, in each of the foregoing embodiments of the present invention, in which a CE is in complex with a ligand, the ligand can be, for example, 4-piperidino-piperidine, tacrine or homatropine.

Accordingly, it is an object of the present invention to provide a three dimensional structure of a mammalian carboxylesterase. The object is achieved in whole or in part by the present invention.

An object of the invention having been stated hereinabove, other objects will be evident as the description proceeds, when taken in connection with the accompanying Drawings and Laboratory Examples as best described hereinbelow.

### BRIEF DESCRIPTION OF THE DRAWINGS

The patent or application file contains at least one drawing executed in color. Copies of this patent or patent application publication with color drawing(s) will be provided by the U.S. Patent and Trademark Office upon request and payment of the necessary fee.

FIG. 1 is a schematic depicting a general two-step activation of the anticancer topoisomerase I poison CPT-11 to SN-38 (an active metabolite) and 4-piperidino-piperidine (4PP) by carboxylesterases.

FIG. 2A is a structure-based sequence alignment of rabbit CE (rCE—-SEQ ID NO: 2), human CE 1 (hCE1—SEQ ID NO: 4) and human intestinal CE (hiCE—SEQ ID NO: 5) obtained with the software ClustalW (Thompson et al., (1994) Nucleic Acids Res. 22: 4673-4680) and refined using the rCE structure. Conserved residues are in black and nonconserved residues are in magenta. Dotted lines indicate missing residues in the rCE structure. N-linked glycosylation sequences, disulfide bonds and putative gate residues are framed in black, and members of the catalytic triad are marked with an asterisk. The catalytic domain is blue; the αβ domain is green; and the regulatory domain is red.

FIG. 2B is a continuation of FIG. 2A and is a structure-based sequence alignment of rabbit CE (rCE—SEQ ID NO: 2), human CE 1 (hCE1—SEQ ID NO: 4) and human intestinal CE (hiCE—SEQ ID NO: 5) obtained with the software ClustalW (Thompson et al., (1994) Nucleic Acids Res. 22: 4673-4680) and refined using the rCE structure. Conserved residues are in black and nonconserved residues are in magenta. Dotted lines indicate missing residues in the rCE structure. N-linked glycosylation sequences, disulfide bonds and putative gate residues are framed in black, and members of the catalytic triad are marked with an asterisk. The catalytic domain is blue; the αβ domain is green; and the regulatory domain is red.

FIG. 3 is a ribbon diagram representing the structure of rabbit liver carboxylesterase indicating the three domains: a catalytic domain, an αβ domain, and a regulatory domain. The catalytic domain is blue; the αβ is green; and the regulatory domain is red. Catalytic residues are in green, N-linked glycosyl groups are in cyan and disulfide linkages are in orange.

FIG. 4 depicts the active site of rCE (green) superimposed on that of two esterases closely related in structure: triacylglycerol hydrolase (PBD entry 1THG; gold) and cholesterol esterase (PDB entry 2BCE; magenta).

FIG. 5 is a stereo view of a composite simulated-annealing omit map (cyan; contoured at 1.0 σ) and the final σ_{A}-weighted (Read, (1986) Acta Crystallog. A 42:140-149) 2Fₒ-F_{c} map (magenta; contoured at 1.0 σ) around the Asn 79 glycosylation site in rCE (both maps at 2.5 Å resolution).

FIG. 6 is a schematic depicting the “side door” binding site for 4PP in rCE. The oligosaccharide chain (cyan) of the Asn 389 glycosylation site comprises three mannoses and two N-acetyl glucosamines (MAN₃NAG₂). The 4PP leaving group of CPT-11 activation (magenta) is stacked in between the indole ring side chain of Trp 550 (yellow) and the proximal NAG (cyan) attached to Asn 389. The catalytic domain is blue; the αβ domain is green; and the regulatory domain is red.

FIG. 7 is a plot depicting the results of CD thermal denaturation studies of wild type rCE performed in the presence of increasing amounts of 4PP. In this figure, solid circles represent denaturation in the presence of 10 mM 4PP, open circles represent denaturation in the presence of 1.6 mM 4PP, solid inverted triangles represent denaturation in the presence of 0.16 mM 4PP, open inverted triangles represent denaturation in the presence of 0.016 mM 4PP and solid squares represent denaturation in the absence of 4PP.

FIG. 8 is a plot depicting the results of thermal denaturation studies of deglycosylated rCE performed in the presence of increasing amounts of 4PP. In this figure, solid circles represent denaturation in the presence of 10 mM 4PP, open circles represent denaturation in the presence of 1.6 mM 4PP, solid inverted triangles represent denaturation in the presence of 0.16 mM 4PP, open inverted triangles represent denaturation in the presence of 0.016 mM 4PP and solid squares represent denaturation in the absence of 4PP.

FIG. 9 is a a plot depicting the melting temperature (Tₘ, ° C.) of wild type (solid circles, solid line) and deglycosylated (open circles, dotted line) rCE in the presence of increasing amounts of 4PP

FIG. 10 is a stereo diagram representing the structural basis of CPT-11 activation by rCE. The regulatory domain is depicted in red.

FIG. 11 is a ribbon diagram depicting the trimeric structure of hCE1. This figure depicts a view of the trimeric structure of hCE1 as viewed down into the active site regions of each monomer. In this figure, each hCE1 monomer is depicted in red, green and blue, with red indicating the regulatory domain, green depicting the (αβ domain and blue depicting the catalytic domain.

FIG. 12 is a diagram depicting the hexameric structure of hCE1 formed in the asymmetric unit. In this figure, each hCE1 monomer is shown in a different color, with helices represented as cylinders and sheets represented as strips.

FIG. 13 is a diagram depicting the binding of tacrine to the active site of hCE1. In this figure, tacrine is depicted as an aqua-colored ball-and-stick model, while hCE1 sidechains that interact with tacrine are depicted in blue, green and magenta.

FIG. 14 is a diagram depicting the binding of homatropine to the active site of hCE1. In this figure, homatropine is depicted as an aqua-colored ball-and-stick model, while hCE1 sidechains that interact with homatropine are depicted in blue, green and magenta.

### BRIEF DESCRIPTION OF THE SEQUENCES IN THE SEQUENCE LISTING

SEQ ID NO: 1 is a DNA sequence encoding a rabbit carboxylesterase polypeptide.

SEQ ID NO: 2 is an amino acid sequence of a rabbit carboxylesterase polypeptide.

SEQ ID NO: 3 is a DNA sequence encoding a human carboxylesterase 1 polypeptide.

SEQ ID NO: 4 is an amino acid sequence of a human carboxylesterase 1 polypeptide.

SEQ ID NO: 5 is an amino acid sequence of a human intestinal carboxylesterase polypeptide.

### BRIEF DESCRIPTION OF THE TABLES

Table 1 is a table depicting the comparison of rCE with related esterases of known structure.

Table 2 is a table summarizing the crystal and data statistics obtained from the crystallized ligand binding domain of rabbit CE in complex with 4-piperidino-piperidine. Data on the unit cell are presented, including data on the crystal space group, unit cell dimensions, molecules per asymmetric cell and crystal resolution.

Table 3 is is a table of the atomic structure coordinate data obtained from X-ray diffraction from the ligand binding domain of rabbit CE in complex with 4-piperidino-piperidine.

Table 4 is a table of the atomic structure coordinate data obtained from X-ray diffraction from acetylcholine esterase isolated from Torpedo californica that was used in the molecular replacement solution of the rabbit CE structure.

Table 5 is a table summarizing the crystal and data statistics obtained from the crystallized ligand binding domain of human CE in complex with tacrine and human CE in complex with homatropine. Data on the unit cell are presented, including data on the crystal space group, unit cell dimensions, molecules per asymmetric cell and crystal resolution

Table 6 is a table of the atomic structure coordinate data obtained from X-ray diffraction from the ligand binding domain of human CE in complex with homatropine.

Table 7 is a table of the atomic structure coordinate data obtained from X-ray diffration from the ligand binding domain of human CE in complex with tacrine.

### DETAILED DESCRIPTION OF THE INVENTION

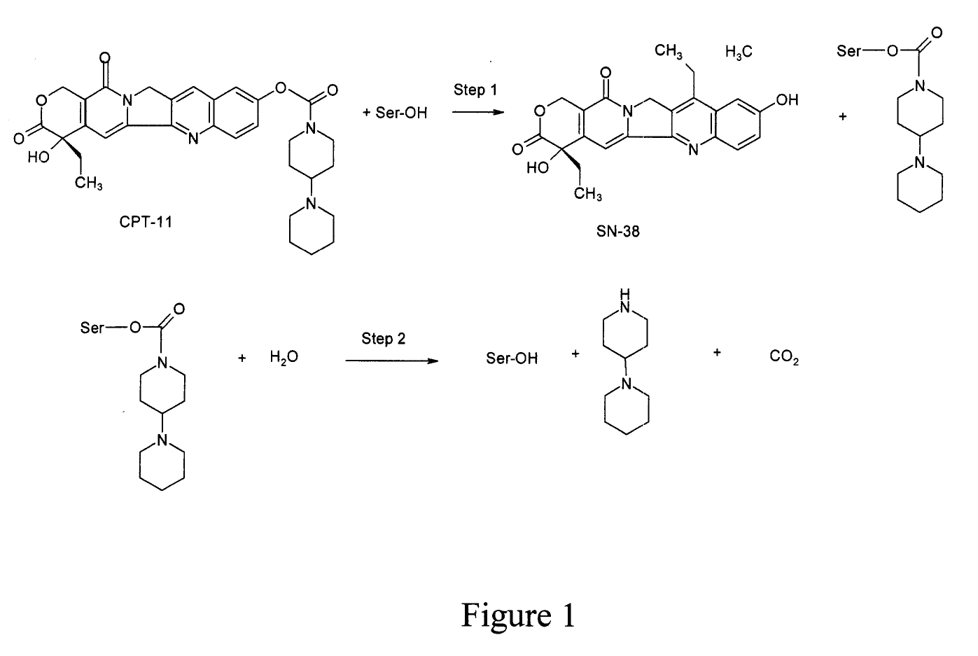

In one aspect, a mammalian carboxylesterase can cleave the anticancer prodrug CPT-11 (irinotecan), a potent topoisomerase I poison, into SN-38, an active metabolite, and 4-piperidino-piperidine (4PP). FIG. 1 depicts a generalized schematic of this process. 4-piperidino-piperidine-carboxylate spontaneously hydrolyzes to 4PP and CO₂ after step 2, as depicted in FIG. 1.

The 2.5 Å crystal structure of rabbit liver carboxylesterase (rCE) is the most efficient enzyme known to activate CPT-11 in this manner, in complex with the leaving group 4PP. 4PP is observed bound adjacent to a high-mannose Asn-linked glycosylation site on the surface of rCE. This product-binding site is separated from the catalytic gorge by a thin wall of amino acid side chains, suggesting that 4PP could be released through this secondary product exit pore. In accordance with the present invention, the crystallographic observation of a leaving group bound on the surface of rCE supports the “back door” product exit site proposed for the acetylcholinesterases. Thus, the present invention facilitates the design of improved anticancer drugs or enzymes for use in viral-directed cancer cotherapies.

Human carboxylesterase 1 plays central roles in several key biological processes. hCE1 comprises of 566 amino acids with two disulfide linkages and one site of asparagine-linked glycosylation. It is related to an hCE1 isoform comprising 568 amino acids in length that contains two amino acids changes, namely an alanine is inserted at position 17, and a glutamine is inserted at position 362.

Thus, in another aspect of the present invention, several crystal structures of human CE in complex with a ligand are provided. More particularly, in one embodiment a 2.4 Å crystal structure of a human CE in complex with tacrine is provided. In another embodiment, a 2.8 Å crystal structure of a human CE in complex with homatropine is provided. These structures can be employed in CE modulator design, which can lead to compounds that can be useful for the treatment of various conditions. For example, a modulator designed using the structures of the present invention can have utility in the treatment of disorders and conditions associated with the biological activity of a CE polypeptide as noted above, including, but not limited to, narcotic metabolism and overdose, CE-based drug-drug interactions, CE-based drug resistance, individualized treatment of disease due to polymorphisms, cancer and other cancer-related disorders, activation of prodrugs, cholesteryl ester formation and hydrolysis, sex hormone maturation, lung surfactant generation, treatment of Alzheimer's Disease, fatty acid ethyl ester formation associated with alcohol abuse, and malaria invasion into human liver.

Until disclosure of the present invention presented herein, the ability to obtain crystalline forms of a mammalian CE has not been realized. And until disclosure of the present invention presented herein, a detailed three-dimensional crystal structure of a mammalian CE polypeptide has not been solved.

In one embodiment, the crystal structure of the carboxylesterase from rabbit liver was determined to 2.54 Å resolution. This structure was determined by crystallizing purified rabbit carboxylesterase, obtaining x-ray diffraction data from these crystals and solving the crystal structure by employing the combined methods of molecular replacement and crystallographic refinement/model building.

The crystal structure of rabbit liver carboxylesterase is of interest for several reasons, including, but not limited to the following. First, this enzyme activates that anticancer drug CPT-11 to SN-38, a potent topoisomerase I poison. Rabbit liver carboxylesterase is the most efficient enzyme known in this activation process. Understanding how rabbit carboxylesterase activates CPT-11 will help elucidate how human enzymes perform this process, which can lead to improved anticancer drugs. Next, rabbit liver carboxylesterase is highly similar in sequence (81% identity at the amino acid level) to the human carboxylesterase 1 that metabolizes cocaine, heroin, many drugs, xenobiotics and organophophorus compounds, and is involved in cholesterol and fatty acid metabolism and hormone production in humans. Further, carboxylesterases are required to activate the cholesterol-lowering statin drugs (e.g., lovastatin). Thus, rabbit liver carboxylesterase can be used to model how these processes occur in humans and to develop improved drugs.

Prior to the present disclosure, the molecular basis for activation of CPT-11 to SN-38 was unknown. In addition, the detailed molecular processes for the breakdown of narcotics, cholesterol and certain hormones by carboxylesterases, were also unknown. This is the first crystal structure of a mammalian carboxylesterase. This structure can be used to analyze how drugs (particularly, but not limited to, CPT-11, cocaine and heroin), cholesterol, fatty acid, hormones, and other xenobiotic compounds are processed in humans.

Additionally, a crystalline structure of the present invention can be used to generate more effective CPT-11 anticancer drugs. Knowledge of how mammals activate CPT-11 to SN-38 by cleaving a carboxylester linkage can facilitate the design of improved CPT-11 analogues that are more easily activated. In addition, the crystal structure of rabbit liver carboxylesterase can facilitate the design of inhibitors to related enzymes (like human butylcholinesterases) that would be useful in reducing the side effects of CPT-11 treatments. Further, this invention can be used to generate inhibitors of carboxylesterases useful in treating narcotic and alcohol overdoses. Inhibitors of carboxylesterases can be given to overdose victims to reduce the metabolism of cocaine and heroin, thus reducing the production of dangerous metabolites.

In addition to providing structural information, crystalline polypeptides provide other advantages. For example, the crystallization process itself further purifies the polypeptide, and satisfies one of the classical criteria for homogeneity. In fact, crystallization frequently provides unparalleled purification quality, removing impurities that are not removed by other purification methods such as HPLC, dialysis, conventional column chromatography, etc. Moreover, crystalline polypeptides are often stable at ambient temperatures and free of protease contamination and other degradation associated with solution storage. Crystalline polypeptides can also be useful as pharmaceutical preparations. Finally, crystallization techniques in general are largely free of problems such as denaturation associated with other stabilization methods (e.g., lyophilization). Once crystallization has been accomplished, crystallographic data provides useful structural information that can assist in the design of compounds that can serve as agonists or antagonists, as described herein below. In addition, the crystal structure provides information useful to map a binding domain, which could then be mimicked by a small non-peptide molecule that would serve as an antagonist or agonist.

### I. Definitions

Following long-standing patent law convention, the terms “a” and “an” mean “one or more” when used in this application, including the claims.

As used herein, the term “about,” when referring to a value or to an amount of mass, weight, time, volume, concentration or percentage is meant to encompass variations of ±20% or ±10%, ±5%, ±1%, or ±0.1% from the specified amount, as such variations are appropriate to perform the disclosed method.

As used herein, the term “mutation” carries its traditional connotation and means a change, inherited, naturally occurring or introduced, in a nucleic acid or polypeptide sequence, and is used in its sense as generally known to those of skill in the art. A nucleic acid or polypeptide in which one or more nucleic acids or amino acids has been substituted for a naturally occurring or non-naturally occurring nucleic acid or amino acid is referred to herein as a “mutant.”

As used herein, the term “labeled” means the attachment of a moiety, capable of detection by spectroscopic, radiologic or other methods, to a probe molecule.

As used herein, the term “target cell” refers to a cell, into which it is desired to insert a nucleic acid sequence or polypeptide, or to otherwise effect a modification from conditions known to be standard in the unmodified cell. A nucleic acid sequence introduced into a target cell can be of variable length. Additionally, a nucleic acid sequence can enter a target cell as a component of a plasmid or other vector or as a naked sequence.

As used herein, the term “transcription” means a cellular process involving the interaction of an RNA polymerase with a gene that directs the expression as RNA of the structural information present in the coding sequences of the gene. The process includes, but is not limited to the following steps: (a) the transcription initiation, (b) transcript elongation, (c) transcript splicing, (d) transcript capping, (e) transcript termination, (f) transcript polyadenylation, (g) nuclear export of the transcript, (h) transcript editing, and (i) stabilizing the transcript.

As used herein, the term “expression” generally refers to the cellular processes by which a polypeptide is produced from RNA.

As used herein, the term “hybridization” means the binding of a probe molecule, for example a molecule to which a detectable moiety has been bound, to a target sample.

As used herein, the term “detecting” means confirming the presence of a target entity by observing the occurrence of a detectable signal, such as a radiologic or spectroscopic signal that will appear exclusively in the presence of the target entity.

As used herein, the term “sequencing” means determining the ordered linear sequence of nucleic acids or amino acids of a DNA or protein target sample, using conventional manual or automated laboratory techniques.

As used herein, the term “isolated” means oligonucleotides substantially free of other nucleic acids, proteins, lipids, carbohydrates or other materials with which they can be associated, such association being either in cellular material or in a synthesis medium. The term can also be applied to polypeptides, in which case the polypeptide will be substantially free of nucleic acids, carbohydrates, lipids and other undesired polypeptides.

As used herein, the term “substantially pure” means that the polynucleotide or polypeptide is substantially free of the sequences and molecules with which it is associated in its natural state, and those molecules used in the isolation procedure. The term “substantially free” means that the sample is at least 50%, 70%, 80% or 90% free of the materials and compounds with which is it associated in nature.

As used herein, the term “primer” means a sequence comprising two or more deoxyribonucleotides or ribonucleotides, for example more than three, more than eight or at least about 20 nucleotides of an exonic or intronic region. Such oligonucleotides are can be, for example, between ten and thirty bases in length.

As used herein, the term “DNA segment” means a DNA molecule that has been isolated free of total genomic DNA of a particular species. In one embodiment, a DNA segment encoding a CE polypeptide refers to a DNA segment that comprises SEQ ID NO: 1 or SEQ ID NO: 3, but can optionally comprise fewer or additional nucleic acids, yet is isolated away from, or purified free from, total genomic DNA of a source species, such as Oryctolagus cuniculus or Homo sapiens. Included within the term “DNA segment” are DNA segments and smaller fragments of such segments, and also recombinant vectors, including, for example, plasmids, cosmids, phages, viruses, and the like.

As used herein, the phrase “enhancer-promoter” means a composite unit that contains both enhancer and promoter elements. An enhancer-promoter is operatively linked to a coding sequence that encodes at least one gene product.

As used herein, the phrase “operatively linked” means that an enhancer-promoter is connected to a coding sequence in such a way that the transcription of that coding sequence is controlled and regulated by that enhancer-promoter. Techniques for operatively linking an enhancer-promoter to a coding sequence are well known in the art; the precise orientation and location relative to a coding sequence of interest is dependent, inter alia, upon the specific nature of the enhancer-promoter.

As used herein, the terms “candidate substance” and “candidate compound” are used interchangeably and refer to a substance that is believed to interact with another moiety, for example a given ligand that is believed to interact with a complete CE polypeptide or a fragment thereof, and which can be subsequently evaluated for such an interaction. Representative candidate substances or compounds include “xenobiotics”, such as drugs and other therapeutic agents, carcinogens and environmental pollutants, natural products and extracts, as well as “endobiotics”, such as steroids. Other examples of candidate compounds that can be investigated using the methods of the present invention include, but are not restricted to, agonists and antagonists of a CE polypeptide, toxins and venoms, viral epitopes, hormones (e.g., opioid peptides, steroids, etc.), hormone receptors, peptides, enzymes, enzyme substrates, co-factors, lectins, sugars, oligonucleotides or nucleic acids, oligosaccharides, proteins, small molecules and monoclonal antibodies.

As used herein, the term “biological activity” means any observable effect flowing from interaction between a CE polypeptide and a ligand. Representative, but non-limiting, examples of biological activity in the context of the present invention include association of a CE with a ligand such as CPT-11 (Irinotecan) and activation of a ligand to another compound, for example activation of CPT-11 to SN-38.

As used herein, the term “modified” means an alteration from an entity's normally occurring state. An entity can be modified by removing discrete chemical units or by adding discrete chemical units. The term “modified” encompasses, but is not limited to, detectable labels as well as those entities added as aids in purification.

As used herein, the terms “structure coordinates” and “structural coordinates” mean mathematical coordinates derived from mathematical equations related to the patterns obtained on diffraction of a monochromatic beam of X-rays by the atoms (scattering centers) of a molecule in crystal form. The diffraction data are used to calculate an electron density map of the repeating unit of the crystal. The electron density maps are used to establish the positions of the individual atoms within the unit cell of the crystal.

Those of skill in the art understand that a set of structure coordinates determined by X-ray crystallography is not without standard error. For the purpose of this invention, any set of structure coordinates for CE or a CE mutant that have a root mean square deviation (RMSD) from ideal of no more than, for example 1.5 Å, no more than 1.0 Å, or no more than 0.5 Å when superimposed, using the polypeptide backbone atoms, on the structure coordinates listed in Tables 3, 6 and 7, shall be considered identical.

As used herein, the term “space group” means the arrangement of symmetry elements of a crystal.

As used herein, the term “molecular replacement” means a method that involves generating a preliminary model of a wild-type CE, or a CE mutant crystal whose structure coordinates are unknown, by orienting and positioning a molecule whose structure coordinates are known within the unit cell of the unknown crystal so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. See, e.g., Lattman, (1985) Method Enzymol., 115: 55-77; The Molecular Replacement Method, Rossmann, (ed.), Gordon & Breach, New York (1972)) By employing the structure coordinates of a mammalian CE provided by the present invention, molecular replacement can be employed to determine the structure coordinates of a crystalline mutant or homologue of a mammalian CE, or of a different crystal form of a mammalian CE.

As used herein, the term “isomorphous replacement” means a method of using heavy atom derivative crystals to obtain the phase information necessary to elucidate the three-dimensional structure of a native crystal (Blundell et al., (1976) Protein Crystallography, Academic Press; Otwinowski, (1991), in Isomorphous Replacement and Anomalous Scattering, (Evans & Leslie, eds.), 80-86, Daresbury Laboratory, Daresbury, United Kingdom). The phrase “heavy-atom derivatization” is synonymous with the term “isomorphous replacement”.

As used herein, the terms “β-sheet” and “beta-sheet” mean the conformation of a polypeptide chain stretched into an extended zig-zig conformation. Portions of polypeptide chains that run “parallel” all run in the same direction. Polypeptide chains that are “antiparallel” run in the opposite direction from the parallel chains.

As used herein, the terms “α-helix” and “alpha-helix” mean the conformation of a polypeptide chain wherein the polypeptide backbone is wound around the long axis of the molecule in a left-handed or right-handed direction, and the R groups of the amino acids protrude outward from the helical backbone, wherein the repeating unit of the structure is a single turnoff the helix, which extends about 0.56 nm along the long axis.

As used herein, the term “unit cell” means a basic parallelepiped shaped block. The entire volume of a crystal can be constructed by regular assembly of such blocks. Each unit cell comprises a complete representation of the unit of pattern, the repetition of which builds up the crystal. Thus, the term “unit cell” means the fundamental portion of a crystal structure that is repeated infinitely by translation in three dimensions. A unit cell is characterized by three vectors a, b, and c, not located in one plane, which form the edges of a parallelepiped. Angles α, β and γ define the angles between the vectors: angle α is the angle between vectors b and c; angle β is the angle between vectors a and c; and angle γ is the angle between vectors a and b. The entire volume of a crystal can be constructed by regular assembly of unit cells; each unit cell comprises a complete representation of the unit of pattern, the repetition of which builds up the crystal.

As used herein, the term “rhombohedral unit cell”, which can alternatively be described as using a hexagonal setting, means a unit cell wherein a=b≠c; and α=β=90°; γ=120°. The vectors a, b and c describe the unit cell edges and the angles α, β, and γ describe the unit cell angles.

As used herein, the term “crystal lattice” means the array of points defined by the vertices of packed unit cells.

As used herein, the term “CE” is used to refer to a carboxylesterase (CE) polypeptide that can bind CPT-11 and/or one or more ligands, and to nucleic acids encoding the same. The term “CE” includes invertebrate homologs; however, CE nucleic acids and polypeptides can also be isolated from vertebrate sources. “CE” further includes vertebrate homologs of CE family members, including, but not limited to, mammalian and avian homologs. Representative mammalian homologs of CE family members include, but are not limited to, rabbit, murine and human homologs.

As used herein, the terms “CE gene product”, “CE protein”, “CE polypeptide”, and “CE peptide” are used interchangeably and mean peptides having amino acid sequences which are substantially identical to native amino acid sequences from an organism of interest and which are biologically active in that they comprise all or a part of the amino acid sequence of a CE polypeptide, or cross-react with antibodies raised against a CE polypeptide, or retain all or some of the biological activity (e.g., ligand binding ability) of the native amino acid sequence or protein. Such biological activity can include immunogenicity.

As used herein, the terms “CE gene product”, “CE protein”, “CE polypeptide”, and “CE peptide” also include analogs of a CE polypeptide. By “analog” is intended that a DNA or peptide sequence can contain alterations relative to the sequences disclosed herein, yet retain all or some of the biological activity of those sequences. Analogs can be derived from genomic nucleotide sequences as are disclosed herein or from other organisms, or can be created synthetically. Those skilled in the art will appreciate that other analogs, as yet undisclosed or undiscovered, can be used to design and/or construct CE analogs. There is no need for a “CE gene product”, “CE protein”, “CE polypeptide”, or “CE peptide” to comprise all or substantially all of the amino acid sequence of a CE polypeptide gene product (e.g. SEQ ID NOs: 2 and 4). Shorter or longer sequences are anticipated to be of use in the invention; shorter sequences are herein referred to as “segments”. Thus, the terms “CE gene product”, “CE protein”, “CE polypeptide”, and “CE peptide” also include fusion, chimeric or recombinant CE polypeptides and proteins comprising sequences of the present invention. Methods of preparing such proteins are disclosed herein and are known in the art.

As used herein, the term “polypeptide” means any polymer comprising any of the 20 protein amino acids, regardless of its size. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide” and “peptide” are used interchangeably herein when referring to a gene product.

As used herein, the term “modulate” means an increase, decrease, or other alteration of any, or all, chemical and biological activities or properties of a wild-type or mutant CE polypeptide. The term “modulation” as used herein refers to both upregulation (i.e., activation or stimulation) and downregulation (i.e. inhibition or suppression) of a response.

As used herein, the terms “CE gene” and “recombinant CE gene” mean a nucleic acid molecule comprising an open reading frame encoding a CE polypeptide of the present invention, including both exon and (optionally) intron sequences.

As used herein, the term “gene” is used for simplicity to refer to a functional protein, polypeptide or peptide encoding unit. As will be understood by those in the art, this functional term includes both genomic sequences and cDNA sequences. Some embodiments of genomic and cDNA sequences are disclosed herein.

As used herein, the term “DNA sequence encoding a CE polypeptide” can refer to one or more coding sequences within a particular individual. Moreover, certain differences in nucleotide sequences can exist between individual organisms, which are called alleles. It is possible that such allelic differences might or might not result in differences in amino acid sequence of the encoded polypeptide yet still encode a protein with the same biological activity. As is well known, genes for a particular polypeptide can exist in single or multiple copies within the genome of an individual. Such duplicate genes can be identical or can have certain modifications, including nucleotide substitutions, additions or deletions, all of which still code for polypeptides having substantially the same activity.

As used herein, the term “intron” means a DNA sequence present in a given gene that is not translated into protein.

As used herein, the term “interact” means detectable interactions between molecules, such as can be detected using, for example, a yeast two-hybrid assay. The term “interact” is also meant to include “binding” interactions between molecules. Interactions can, for example, be protein-protein or protein-nucleic acid in nature.

As used herein, the terms “cells,” “host cells,” and “recombinant host cells” are used interchangeably and mean not only the particular subject cell, but also to the progeny or potential progeny of such a cell. Because certain modifications can occur in succeeding generations due to either mutation or environmental influences, such progeny might not, in fact, be identical to the parent cell, but are still included within the scope of the term as used herein. The terms can encompass a “target cell” as defined herein. For example, a recombinant host cell or a host cell can also be a target cell, into which a nucleic acid can be introduced.

As used herein, the term “agonist” means an agent that supplements or potentiates the bioactivity of a functional CE gene or protein, or that supplements or potentiates the bioactivity of a naturally occurring or engineered non-functional CE gene or protein.

As used herein, the term “antagonist” means an agent that decreases or inhibits the bioactivity of a functional CE gene or protein, or that decreases or inhibits the bioactivity of a naturally occurring or engineered non-functional CE gene or protein.

As used herein, the terms “chimeric protein” or “fusion protein” are used interchangeably and mean a fusion of a first amino acid sequence encoding a CE polypeptide with a second amino acid sequence defining a polypeptide domain foreign to, and not homologous with, any domain of one of a CE polypeptide. A chimeric protein can present a foreign domain that is found in an organism that also expresses the first protein, or it can be an “interspecies” or “intergenic” fusion of protein structures expressed by different kinds of organisms. In general, a fusion protein can be represented by the general formula X—CE—Y, wherein CE represents a portion of the protein which is derived from a CE polypeptide, and X and Y are independently absent or represent amino acid sequences which are not related to a CE sequence in an organism, which includes naturally occurring mutants. The term “chimeric gene” refers to a nucleic acid construct that encodes a “chimeric protein” or “fusion protein” as defined herein.

As used herein, the term “therapeutic agent” is a chemical entity intended to effectuate a change in an organism. An organism can be, but is not required to be, a human being. It is not necessary that a therapeutic agent be known to effectuate a change in an organism; chemical entities that are suspected, predicted or designed to effectuate a change in an organism are therefore encompassed by the term “therapeutic agent.” The effectuated change can be of any kind, observable or unobservable, and can include, for example, a change in the biological activity of a protein.

Representative therapeutic compounds include small molecules, proteins and peptides, oligonucleotides of any length, “xenobiotics”, such as drugs and other therapeutic agents, carcinogens and environmental pollutants, natural products and extracts, as well as “endobiotics”, such as steroids, fatty acids and prostaglandins. Other examples of therapeutic agents can include, but are not restricted to, agonists and antagonists of a CE polypeptide, toxins and venoms, viral epitopes, hormones (e.g., opioid peptides, steroids, etc.), hormone receptors, peptides, enzymes, enzyme substrates, co-factors, lectins, sugars, oligonucleotides or nucleic acids, oligosaccharides, proteins, small molecules and monoclonal antibodies.

### II. General Considerations

Mammalian carboxylesterases (CEs) are important to the metabolism and detoxification of numerous endogenous and xenobiotic compounds (Williams, (1985) Clin. Pharmacokinet. 10: 392-403). They also play a role in the activation of prodrugs in humans. Prodrugs containing ester linkages can increase the solubility and bio-availability of therapeutic agents. Bodor & Buchwald, (2000) Med. Res. Rev. 20: 58-101. The promiscuous mammalian CEs act on a wide variety of ester, amide and thioester substrates (Williams, (1985) Clin. Pharmacokinet. 10: 392-403) and are known to metabolize numerous analgesic and narcotic compounds, including aspirin (Joly & Brown, (1986) Toxicol. Appl. Pharmacol. 84: 523-532), cocaine (Brzezinski et al., (1997) Drug Metab. Dispos. 25: 1089-1096), heroin (Kamendulis et al., (1996) J. Pharmacol. Exp. Ther. 279: 713-717), procaine (Joly & Brown, (1986) Toxicol. Appl. Pharmacol. 84: 523-532) and meperidine (Lotti et al., (1983) Biochem. Pharmacol. 32: 3735-3738). Esterases, including CEs, share a common structural framework, active site and two-step serine hydrolase mechanism. Ollis et al., (1992) Protein Eng. 5: 197-211. The active site typically contains a serine hydrolase catalytic triad, which is composed of a Ser, a His and either an Asp or a Glu residue.

Varying levels of activation of the anticancer prodrug CPT-11 (irinotecan, 7-ethyl-10-[4-(1-piperidino)-1-piperidino] carbonyloxy-camptothecin) have been observed across species, with the highest levels observed in rodents. Morton et al., (2000) Cancer Res. 60: 4206-4210. A rabbit liver CE (rCE) was recently found to be the most efficient enzyme identified to date in the activation of CPT-11. Potter et al., (1998) Cancer Res. 58: 2646-2651. Human liver CE-1, the human homolog of rCE (81% sequence identity), is unable to process CPT-11. Danks et al., (1999) Clin. Cancer Res. 5: 917-924. However, human intestinal CE (hiCE), which shares only 47% sequence identity with rCE, is able to activate CPT-11 efficiently. Khana et al., (2000) Cancer Res. 60: 4725-4728. hiCE differs by only six amino acids from liver human CE-2 (hCE2), which can also activate CPT-11. Pindel et al., (1997) J. Biol. Chem. 272: 14769-14775.

In vivo, CEs activate the prodrug CPT-11 via cleavage to form SN-38. Chabot, (1997) Clin. Pharmacokinet. 33: 245-259. SN-38 is a potent topoisomerase I-specific poison, which traps covalent topoisomerase I-DNA complexes, causing a toxic accumulation of double-strand DNA breaks in actively dividing cancer cells. CPT-11 has been approved for use in the treatment of colon cancer and is now being assessed for activity against a variety of other solid tumors. Activation of CPT-11 by CE proceeds via a two-step serine hydrolase mechanism involving an acyl-enzyme intermediate (see FIG. 1). Typically, only ˜2% of the SN-38 generated by the activation of CPT-11 makes it to the tumor in humans; hence, developing a more effective way to deliver SN-38 to solid malignancies is of interest.

Expression of rCE in human tumor cell lines and in xenografts grown in immune-deprived mice sensitizes them to CPT-11. Potter et al., (1998) Cancer Res. 58: 2646-2651; Danks et al., (1999) Clin. Cancer Res. 5: 917-924; Danks et al., (1998) Cancer Res. 58: 20-22; Potter et al., (1998) Cancer Res. 58: 3627-3632. Viral-based gene therapy approaches have also demonstrated promise for providing an efficient, targeted way to activate CPT-11 in humans. Weirdl et al., (2001) Cancer Res. 61: 5078-5082; Meck et al., (2001) Cancer Res. 61: 5083-5089. For example, adenoviruses expressing rCE can sensitize tumor cells to CPT-11 up to 127-fold, and a secreted form of the protein can elicit a bystander effect to cells not expressing the enzyme. Weirdl et al., (2001) Cancer Res. 61: 5078-5082. Additionally, ex vivo purging approaches to eliminate neuroblastoma cells from bone marrow have been designed and are now being tested for clinical utility. Meck et al., (2001) Cancer Res. 61: 5083-5089. Ultimately, rCE could prove useful in sensitizing human tumors to CPT-11 or other ester-linked prodrugs. Thus, an aspect of the present invention is to provide the first structural view of a mammalian carboxylesterase and insights into CPT-11 activation.

Human carboxylesterase 1 plays central roles in several key biological processes. hCE1 comprises of 566 amino acids with two disulfide linkages and one site of asparagine-linked glycosylation. It is related to an hCE1 isoform comprising 568 amino acids in length that contains two amino acids changes, namely an alanine is inserted at position 17, and a glutamine is inserted at position 362.

Thus, in another aspect of the present invention, several crystal structures of human CE in complex with a ligand are provided. More particularly, in one embodiment a 2.4 Å crystal structure of a human CE in complex with tacrine is provided. In another embodiment, a 2.8 Å crystal structure of a human CE in complex with homatropine is provided. These structures can be employed in CE modulator design, which can lead to compounds that can be useful for the treatment of various conditions. For example, a modulator designed using the structures of the present invention can have utility in the treatment of disorders and conditions associated with the biological activity of a CE polypeptide as noted above, including, but not limited to, CE-based drug-drug interactions, CE-based drug resistance, individualized treatment of disease due to polymorphisms, cancer and other cancer-related disorders, activation of prodrugs, cholesteryl ester formation and hydrolysis, sex hormone maturation, lung surfactant generation, treatment of Alzheimer's Disease, fatty acid ethyl ester formation associated with alcohol abuse, and malaria invasion into human liver.

### III. Production of CE Polypeptides

The native and mutated CE polypeptides, and fragments thereof, of the present invention can be chemically synthesized in whole or part using techniques that are well-known in the art (see, e.g., Creighton, (1983) Proteins: Structures and Molecular Principles, W. H. Freeman & Co., New York, incorporated herein in its entirety). Alternatively, methods that are well known to those skilled in the art can be used to construct expression vectors containing a partial or the entire native or mutated CE polypeptide coding sequence and appropriate transcriptional/translational control signals. These methods include in vitro recombinant DNA techniques, synthetic techniques and in vivo recombination/genetic recombination (see, e.g., the techniques described in Sambrook et al., (1989) Molecular Cloning: A Laboratory Manual, Cold Spring Harbor Laboratory, New York, and Ausubel et al., (1989) Current Protocols in Molecular Biology, Greene Publishing Associates and Wiley Interscience, New York, both incorporated herein in their entirety).

A variety of host-expression vector systems can be employed to express a CE coding sequence. These include but are not limited to microorganisms such as bacteria transformed with recombinant bacteriophage DNA, plasmid DNA or cosmid DNA expression vectors containing a CE coding sequence; yeast transformed with recombinant yeast expression vectors containing a CE coding sequence; insect cell systems infected with recombinant virus expression vectors (e.g., baculovirus) containing a CE coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing a CE coding sequence; or animal cell systems. The expression elements of these systems vary in their strength and specificities.

Depending on the host/vector system utilized, any of a number of suitable transcription and translation elements, including constitutive and inducible promoters, can be used in the expression vector. For example, when cloning in bacterial systems, inducible promoters such as pL of bacteriophage λ, plac, ptrp, ptac (ptrp-lac hybrid promoter) and the like can be used. When cloning in insect cell systems, promoters such as the baculovirus polyhedrin promoter can be used. When cloning in plant cell systems, promoters derived from the genome of plant cells, such as heat shock promoters; the promoter for the small subunit of RUBISCO; the promoter for the chlorophyll a/b binding protein) or from plant viruses (e.g., the 35S RNA promoter of CaMV; the coat protein promoter of TMV) can be used. When cloning in mammalian cell systems, promoters derived from the genome of mammalian cells (e.g., metallothionein promoter) or from mammalian viruses (e.g., the adenovirus late promoter; the vaccinia virus 7.5K promoter) can be used. When generating cell lines that contain multiple copies of the tyrosine kinase domain DNA, SV40-, BPV- and EBV-based vectors can be used with an appropriate selectable marker.

### IV. Formation of CE Crystals

In one embodiment, the present invention provides crystals of a rabbit CE (rCE). The crystals were obtained using the methodology disclosed in the Laboratory Examples. The rCE crystals, which can be native crystals, derivative crystals or co-crystals, have rhombohedral unit cells, (a rhombohedral unit cell, which can alternatively be described using a hexagonal setting, is a unit cell wherein a=b≠c, and wherein α=β=90; γ=120°) and space group symmetry R32. In one embodiment, there is one rCE molecule in the asymmetric unit. In the rCE crystalline form, the unit cell has dimensions of a=b=110.23 Å, c=282.52 Å, and α=β=90; γ=120°.

In another embodiment, human CE1 (hCE1) crystals are provided. The hCE1 crystals, which can also be native crystals, derivative crystals or co-crystals, have monoclinic unit cells (a monoclinic unit cell is a unit cell wherein a≠b≠c, and wherein α=γ=90°; β≠90°) and space group symmetry of P2₁. In one embodiment, there are six hCE1 molecules in the asymmetric unit. In one hCE1 crystalline form, the unit cell has dimensions of a=90 Å, b=117 Å, c=176 Å, and α=γ=90°; β=95.7°. In another hCE1 crystalline form, the unit cell has dimensions of a=55.4 Å, b=178.8 Å, c=199.6 Å, and α=γ=90°; β=90.2°. These crystals can also be formed by following the methodology disclosed in the Laboratory Examples.

The structure of the 4PP-bound form of rCE was solved by molecular replacement using the structure of the Torpedo californica acetylcholine esterase as a search model (Table 4; RCSB Protein ID No. 1ACE; available online at http://www.rcsb.org/pdb/). The ligand-bound form of rCE was refined to a resolution of about 2.54 Å. The structure of the tacrine-bound form of hCE1 was solved by molecular replacement using the rCE structure (Table 3) as a search model and refined to a resolution of about 2.4 Å. The structure of the homatropin-bound form of hCE1 was solved by molecular replacement using the rCE structure (Table 3) as a search model and refined to a resultion of about 2.8 Å.

A heavy atom derivatized form of a CE-ligand structure can be solved using single isomorphous replacement anomalous scattering (SIRAS) techniques and/or multiwavelength anomalous diffraction (MAD) techniques. In the SIRAS method of solving protein crystals, a derivative crystal is prepared that contains an atom that is heavier than the other atoms of the sample. Heavy atom derivative crystals are commonly prepared by soaking a crystal in a solution containing a selected heavy atom salt. For example, some heavy atom derivative crystals have been prepared by soaking a crystalline form of the protein of interest in a solution of methyl mercury chloride (MeHgCl).

A representative heavy atom that can be incorporated into a derivative crystal is iodine. Heavy atoms can associate with the protein of interest, or can be localized in a ligand that associates with a protein of interest. Thus, in a heavy atom derivative, a crystalline form can optionally contain one or more atoms having an atomic weight of 40 grams/mol or greater.

Analysis of derivative crystals takes advantage of differences in the reflections from the derivative crystal as compared to the underivatized crystal. Symmetry-related reflections in the X-ray diffraction pattern, which are usually identical, are altered by the anomalous scattering contribution of the heavy atoms. The measured differences in symmetry-related reflections are used to determine the position of the heavy atoms, leading to an initial estimation of the diffraction phases, and subsequently, an electron density map is prepared. The prepared electron density map is then used to identify the position of the other atoms in the sample.

IV.A. Preparation of CE Crystals

The native and derivative co-crystals, and fragments thereof, disclosed in the present invention can be obtained by employing a variety of techniques, including batch, liquid bridge, dialysis, vapor diffusion and sitting drop methods (see, e.g., McPherson, (1982) Preparation and Analysis of Protein Crystals, John Wiley, New York.; McPherson, (1990) Eur. J. Biochem. 189:1-23.; Weber, (1991) Adv. Protein Chem. 41:1-36). Optionally, the vapor diffusion and sitting drop methods are used for the crystallization of CE polypeptides and fragments thereof.

In general, native crystals of the present invention are grown by dissolving substantially pure CE polypeptide or a fragment thereof in an aqueous buffer containing a precipitant at a concentration just below that necessary to precipitate the protein. Water is removed by controlled evaporation to produce precipitating conditions, which are maintained until crystal growth ceases.

In one embodiment of the invention, native crystals are grown by vapor diffusion (see, e.g., McPherson, (1982) Preparation and Analysis of Protein Crystals, John Wiley, New York; McPherson, (1990) Eur. J. Biochem. 189:1-23). In this method, the polypeptide/precipitant solution is allowed to equilibrate in a closed container with a larger aqueous reservoir having a precipitant concentration optimal for producing crystals. Generally, less than about 25 μL of CE polypeptide solution is mixed with an equal volume of reservoir solution, giving a precipitant concentration about half that required for crystallization. This solution is suspended as a droplet underneath a coverslip, which is sealed onto the top of the reservoir. The sealed container is allowed to stand, until crystals grow. Crystals generally form within two to six weeks, and are suitable for data collection within approximately seven to ten weeks. Of course, those of skill in the art will recognize that the above-described crystallization procedures and conditions can be varied.

IV.B. Preparation of Derivative Crystals

Derivative crystals of the present invention, e.g. heavy atom derivative crystals, can be obtained by soaking native crystals in mother liquor containing salts of heavy metal atoms (e.g., one or more atoms having an atomic weight of 40 grams/mol or greater). Alternatively, a ligand comprising a heavy atom can be associated with a protein, and subsequently co-crystallized. Such derivative crystals are useful for phase analysis in the solution of crystals of the present invention. This mechanism provides derivative crystals suitable for use as isomorphous replacements in determining the X-ray crystal structure of a CE polypeptide. Additional reagents useful for the preparation of the derivative crystals of the present invention will be apparent to those of skill in the art after review of the disclosure of the present invention presented herein.

IV.C. Preparation of Co-Crystals

Co-crystals of the present invention can be obtained by soaking a native crystal in mother liquor containing compounds known or predicted to bind a CE polypeptide, or a fragment thereof. Alternatively, co-crystals can be obtained by co-crystallizing a CE polypeptide or a fragment thereof in the presence of one or more compounds known or predicted to bind the polypeptide, or that are known to generate enzymatic cleavage products that are known or suspected to associate with a CE. In one embodiment of the present invention, for example, the ligand CPT-11 or the ligand 4PP (an enzymatic cleavage product of the activation of CPT-11 to SN-38), or an iodinated form of these ligands, is co-crystallized with a CE-ligand complex. In other embodiments, tacrine or homatropine is co-crystallized with a CE-ligand complex.

IV.D. Solving a Crystal Structure of the Present Invention

Crystal structures of the present invention can be solved by employing a variety of techniques including, but not limited to, isomorphous replacement anomalous scattering or molecular replacement methods. Computer software packages will also be helpful in solving a crystal structure of the present invention. Applicable software packages include but are not limited to X-PLOR™ program (Brünger, (1992) X-PLOR, Version 3.1. A System for X-ray Crystallography and NMR, Yale University Press, New Haven, Conn.; X-PLOR is available from Accelrys, San Diego, Calif.), Xtal View (McRee, (1992) J. Mol. Graphics 10: 44-47; X-tal View is available from the San Diego Supercomputer Center), SHELXS 97 (Sheldrick (1990) Acta Cryst. A46: 467; SHELX 97 is available from the Institute of Inorganic Chemistry, Georg-August-Universität, Göttingen, Germany), HEAVY, version 4.5 (Terwilliger, Los Alamos National Laboratory) and SHAKE-AND-BAKE (Hauptman, (1997) Curr. Opin. Struct. Biol. 7: 672-80; Weeks et al., (1993) Acta Cryst. D49: 179; available from the Hauptman-Woodward Medical Research Institute, Buffalo, N.Y.) can be used. See also, Ducruix & Geige, (1992) Crystallization of Nucleic Acids and Proteins: A Practical Approach, IRL Press, Oxford, England, and references cited therein.

IV.E. Generation of Easily-Solved CE Crystals

The present invention discloses a substantially pure CE polypeptide in crystalline form. In some embodiments, CE is crystallized with a bound ligand. Crystals are formed from CE polypeptides that can be expressed by a cell culture, such as E. coli or insect (e.g. Sf21) cells, but can also be isolated from tissue, such as rabbit liver or human brain or intestine, for example. Bromo- and iodo-substitutions can be made during the preparation of crystal forms and can act as heavy atom substitutions in CE ligands and in crystals of CE. This method can be advantageous for the phasing of the crystal, which is a crucial, and sometimes limiting, step in solving the three-dimensional structure of a crystallized entity. Thus, the need for generating the heavy metal derivatives traditionally employed in crystallography might be eliminated. After the three-dimensional structure of a CE with or without a ligand bound is determined, the resultant three-dimensional structure can be employed in computational methods to design synthetic ligands for CE and other CE polypeptide fragments. Further activity structure relationships can be determined through routine testing, using assays disclosed herein and known in the art.

IV.F. Ligand

In one aspect of the present invention, a molecule of 4-piperidino-piperidine (4PP) was co-crystallized with rCE. 4PP is a product of the enzymatic cleavage of the cancer prodrug CPT-11, the generic form of which is referred to as irinotecan (7-ethyl-10-[4-(1-piperidino)-1-piperidino]carbonyl camptothecin). Irinotecan has the chemical structure:

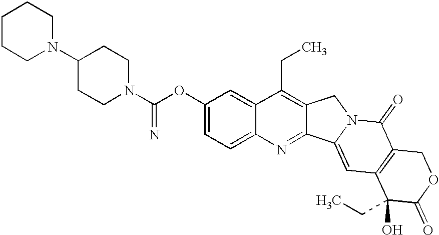

and is a DNA topoisomerase inhibitor. Irinotecan is a semisynthetic derivative of the compound camptothecin.

Cleavage (i.e. activation) of irinotecan by CE gives carbon dioxide, 4-piperidino-piperidine, which co-crystallized with CE and has the chemical structure:

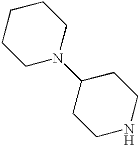

and SN-38, which has the chemical structure:

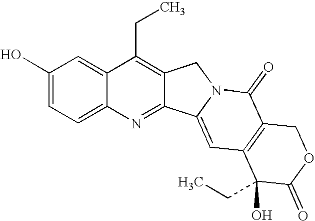

The preparation of irinotecan has been previously described (U.S. Pat. No. 4,604,463 and Sawada et al., (1991) Chem. Pharm. Bull. 39:1446) and its de-esterification to its active metabolite has also been described (Kaneda et al., (1990) Cancer Res. 50:1715). Both irinotecan and 4-piperidino-piperidine are commercially available, irinotecan under the trade name Camptosar® from Pharmacia Corporation of Peapack, N.J., United States of America and 4-piperidino-piperidine from Sigma Chemical Company of St. Louis, Mo., United States of America.

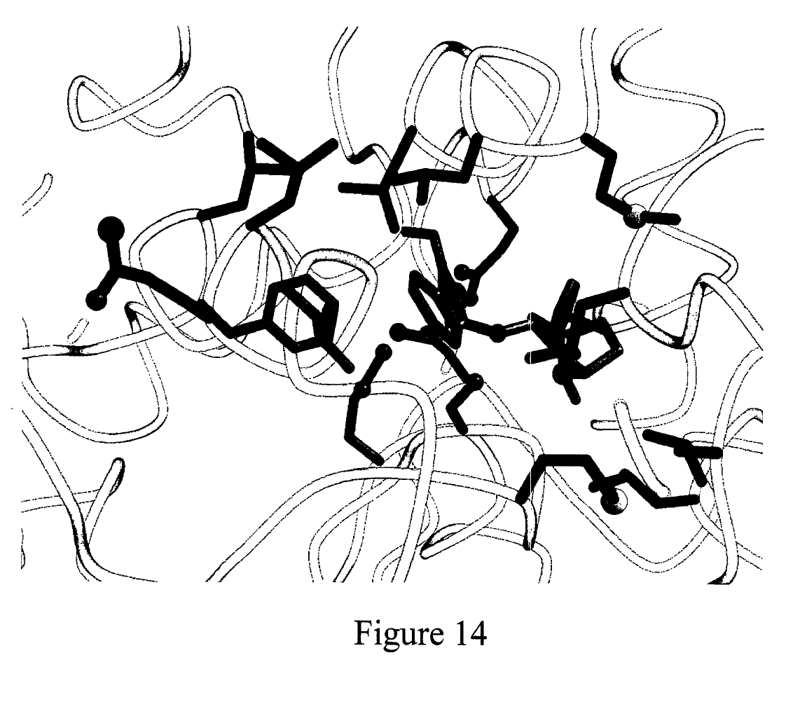

In another embodiment, hCE1 was co-crystallized with the ligand homotropine (endo-(±)-α-hydroxybenzeneacetic acid 8-methyl-8-azabicyclo[3.2.1]oct-3-yl ester) (see FIG. 14). In FIG. 14, homatropine is an aqua-colored ball-and-stick model, while hCE1 sidechains that interact with homatropine are depicted in blue, green and magenta.

Homatropine can be synthetically prepared (see, e.g., Chemnitius, (1927) J. Prakt. Chem. 117: 142). Homatropine hydrobromide is commercially available from Sigma Chemical Company. Homatropine has the chemical structure:

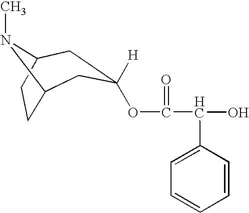

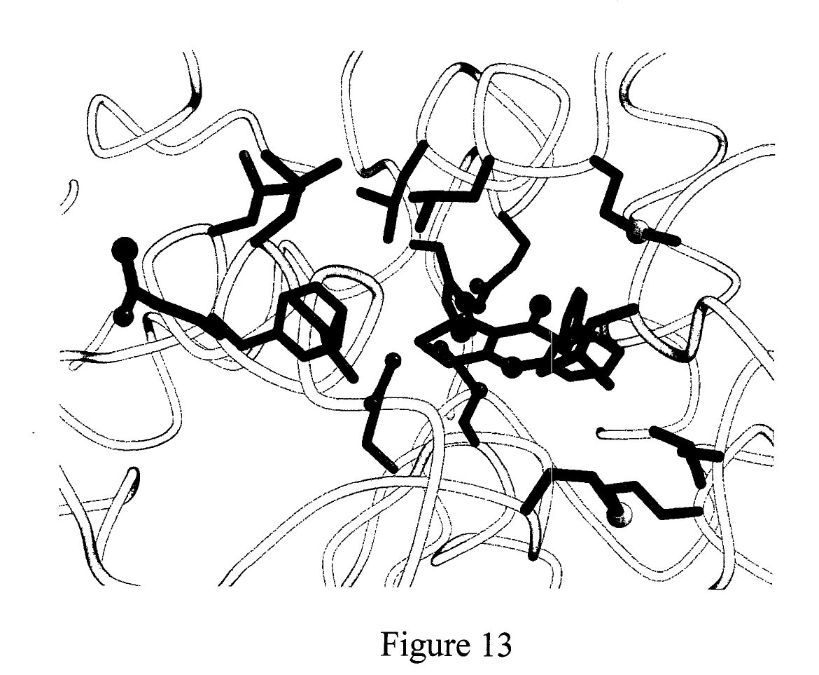

In yet another embodiment, hCE1 was co-crystallized with the ligand tacrine (1,2,3,4-tetrahydro-9-acridinamine) (see FIG. 13). In FIG. 13, tacrine is an aqua-colored ball-and-stick model. hCE1 sidechains that interact with tacrine are depicted in blue, green and magenta.

Tacrine can be synthetically prepared (see, e.g. Albert & Gledhill, (1945) J. Chem. Soc. Ind. 64: 169T) and is commercially available under the trade name Cognex® from First Horizon Pharmaceutical Corporation, Roswell, Ga., United States of America. Tacrine has the chemical structure

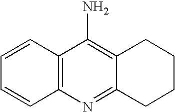

### V. Uses of CE Crystals and the Three-Dimensional Structure of CE

The rCE1 and hCE1 structures of the present invention can be employed in a range of applications. In humans, CE1 is expressed and/or identified in several human tissues and is involved in a wide array of key biological processes. The hCE1 and rCE1 structures of the present invention can be employed in the rational design of modulators of these processes or can be employed in structural studies to understand these conditions. Some of the identified roles of hCE1 follow. In some embodiments of the present invention, modulators of these processes can be designed and synthesized.

It is known that hCE1 plays a critical role in the breakdown of drugs and other harmful compounds in the liver, small intestine, kidneys, brain, lung (e.g., via alveolar macrophages/monocytes) and circulating plasma. Drugs metabolized by hCE1 include cocaine, heroine, meperidine, and lidocaine. Toxic xenobiotics metabolized by hCE1 include organophosphates.

hCE1 is also known to activate several key prodrugs (i.e. drugs that must be processed in patients to their active form) in the liver, small intestine, kidneys, brain, lung and circulating plasma. Prodrugs activated by hCE1 include HMG-CoA reductase inhibitor lovastatin used for high cholesterol, the angiotension-converting enzyme (ACE) inhibitors temocapril, delapril and imidapril used to treat hypertension. Interestingly, hCE1, which shares 81% sequence identity with rCE (the structure of which forms an aspect of the present invention), does not efficiently activate the anticancer drug CPT-11. Thus, modulating hCE1 activity can lead to different and desirable prodrug activation profiles.

Several reports have indicated that hCE1 is capable of transferring fatty acids from fatty acyl-CoA to cholesterol to generate cholesteryl esters, which are vital to cholesterol trafficking both in cells and systemically. This reaction is termed an acyl coenzyme A: cholesterol acyltransferase (ACAT) activity. In addition, it has been suggested that hCE1 also hydrolyzes cholesteryl esters to free fatty acids and cholesterol; again, this action is critical to cholesterol trafficking. The sum effect of this role in humans is to assist with cholesterol transport, protect against atherosclerosis, and to assist with normal heart and liver function. These actions are associated with hCE1 present or expressed in liver, heart and circulating plasma. Another application of the structures of the present invention, therefore, can be in the area of modulating cholesterol trafficking.

hCE1 also plays a role in the addition of fatty acids to testosterone in the testes. This action allows the hormone to be circulated and trafficked properly in the body and the structures of the present invention can also play a role in the development of modulators of testosterone synthesis.

Further, hCE1 appears to be involved in the generation of lung surfactant, a phospholipid and protein mixture necessary for normal lung function and defense against xenobiotics. This action is related to expression in the lung, as well as alveolar macrophages and monocytes. In premature infants, respiratory distress syndrome (RDS) is a leading complication and is often fatal. This condition is caused by the lack of necessary levels of lung surfactant. Thus, modulation of lung surfactant generation might facilitate the development of a treatment for RDS.

The pathology of Alzheimer's Disease (AD) appears to require the presence of high levels of cholesteryl esters in brain. As noted above, hCE1 can generate cholesteryl esters via its ACAT activity. Thus, inhibiting hCE1 in human brain might lead to a lessening of the progression of AD. Indeed, the first approved treatment of AD, tacrine, was originally thought to only inhibit acetylcholinesterase (AcChE), thus lengthening the lifetime of the neurotransmitter acetylcholine. In one aspect of the present invention, it is demonstrated that tacrine binds effectively to hCE1. Thus, tacrine might have an additional, as yet unidentified, effect in the brains of AD patients. The structures of the present invention can, therefore, be employed in developing tacrine analogs and other hCE1 modulators that could be employed in the treatment of AD.

hCE1 also catalyzes the generation of fatty acid ethyl esters (FAEE), which are cytotoxic nonoxidative byproducts of alcohol abuse. This action is associated with hCE1 expression in liver, heart and brain. FAEEs uncouple oxidative phosphorylation in mitochondria, inhibit protein synthesis and cell proliferation, and increase lysosome fragility. FAEEs are present in adipose tissue, liver, pancreas and heart during acute alcohol intoxication. However, as a consequence of chronic abuse, they can build to toxic levels in adipose and heart tissue. Thus, modulation of FAEE production via modulation of hCE1 activity might form an aspect of a treatment for alcohol abuse.

Malaria is the number one health problem in the world, causing two million deaths per year. The sporozoite stage of the life cycle of Plasmodium falciparum, the parasite that causes malaria, resides in human liver as a necessary part of parasite infection. Malaria sporozoites must adhere to human liver cells before they can enter the cells. hCE1 is one of two proteins on the surface of human hepatocytes that are specifically contacted by the malaria surface sporozoite CS and/or TRAP proteins. This action is related to hCE1 expressed on the surface of human liver cells, a stage of hCE1 cycling prior to its secretion into the plasma. Thus, hCE1 plays a role in the life cycle and pathogenesis of the malaria parasite, and modulation of hCE1 activity and/or structure, using the present disclosure as a guide, might be employed as a component in a malaria treatment regimen.

V.A. Design and Development of CE Modulators

The knowledge of the structure of a rabbit CE and a human CE, aspects of the present invention, provides a tool for investigating the mechanism of action of CE polypeptides in a subject. For example, various computer models, as described herein, can predict the binding of various substrate molecules to a mammalian CE, for example a rabbit CE (e.g., rCE) or a human CE (e.g., hCE1). Upon discovering that such binding in fact takes place, knowledge of the protein structure then allows design and synthesis of small molecules that mimic the functional binding of the substrate to a rabbit CE (e.g., rCE) or a human CE (e.g., hCE1). This is the method of “rational” drug design, further described herein.

Use of the isolated and purified a rabbit CE (e.g., rCE) or a human CE (e.g., hCE1) crystalline structures of the present invention in rational drug design is thus provided in accordance with the present invention. Additional rational drug design techniques are described in U.S. Pat. Nos. 5,834,228 and 5,872,011, incorporated herein in their entirety.

Thus, in addition to the compounds described herein, other sterically similar compounds can be formulated to mimic the key structural regions of CEs in general, mammalian CEs in particular, and more particularly rCE or hCE1. The generation of a structural functional equivalent can be achieved by the techniques of modeling and chemical design known to those of skill in the art and described herein. It will be understood that all such sterically similar constructs fall within the scope of the present invention.

V.A.1. Rational Drug Design

The three-dimensional structure of a mammalian CE is unprecedented and will greatly aid in the development of new synthetic ligands for a CE polypeptide, such as CE agonists and antagonists, including those that bind exclusively to any one of the CE orthologs and/or subtypes. In addition, CE is well suited to modern methods, including three-dimensional structure elucidation and combinatorial chemistry, such as those disclosed in U.S. Pat. No. 5,463,564, incorporated herein by reference.

Computer programs that utilize crystallography data when practicing the present invention will enable the rational design of ligands to these receptors. Programs such as RASMOL (Biomolecular Structures Group, Glaxo Wellcome Research & Development Stevenage, Hertfordshire, UK Version 2.6, August 1995, Version 2.6.4, December 1998, Copyright © Roger Sayle 1992-1999) can use the atomic structural coordinates from crystals of the present invention, the atomic structural coordinates from crystals generated by practicing the invention or the atomic structural coordinates from crystals used to practice the invention by generating three-dimensional models and/or determining the structures involved in ligand binding. Computer programs such as those sold under the registered trademark INSIGHT II® and such as GRASP (Nicholls et al., (1991) Proteins 11: 281-96) allow for further manipulations and the ability to introduce new structures. In addition, high throughput binding and bioactivity assays can be devised using purified recombinant protein in order to refine the activity of a designed ligand.

A method of identifying modulators of the activity of a CE polypeptide using rational drug design is thus provided in accordance with the present invention. In one embodiment, the method comprises designing a potential modulator for a CE polypeptide of the present invention that will form non-covalent bonds with amino acids in a ligand binding cavity based upon the crystalline structure of a CE polypeptide; synthesizing the modulator; and determining whether the potential modulator modulates the activity of the CE polypeptide. The determination of whether the modulator modulates the biological activity of a CE polypeptide is made in accordance with the screening methods disclosed herein, or by other screening methods known to those of skill in the art. Modulators can be synthesized using techniques known to those of ordinary skill in the art.

In an alternative embodiment, a method of designing a modulator of a CE polypeptide in accordance with the present invention is disclosed comprising: (a) selecting a candidate CE ligand; (b) determining which amino acid or amino acids of a CE polypeptide interact with the ligand using a three-dimensional model of a crystallized CE; (c) identifying in a biological assay for CE activity a degree to which the ligand modulates the activity of the CE polypeptide; (d) selecting a chemical modification of the ligand wherein the interaction between the amino acids of the CE polypeptide and the ligand is predicted to be modulated by the chemical modification; (e) performing the chemical modification on the ligand to form a modified ligand; (f) contacting the modified ligand with the CE polypeptide; (g) identifying in a biological assay for CE activity a degree to which the modified ligand modulates the biological activity of the CE polypeptide; and (h) comparing the biological activity of the CE polypeptide in the presence of modified ligand with the biological activity of the CE polypeptide in the presence of the unmodified ligand, whereby a modulator of a CE polypeptide is designed.

V.A.2. Use of CE Structural Coordinates for Molecular Design

For the first time, the present invention permits the use of molecular design techniques to design, select and synthesize chemical entities and compounds, including modulatory compounds, which are capable of binding to the ligand binding cavity or an accessory binding site of a CE (including but not limited to a mammalian CE, such as rCE or hCE1), in whole or in part. Correspondingly, the present invention also provides for the application of similar techniques in the design of modulators of any CE polypeptide.

In accordance with an embodiment of the present invention, the structure coordinates of a crystalline rCE or a crystalline hCE1 can be used to design compounds that bind to a CE and alter the properties of a CE (for example, ligand binding ability) in different ways. One aspect of the present invention provides for the design of compounds that act as competitive inhibitors of a CE polypeptide by binding to all, or a portion of, the binding sites on a CE. The present invention also provides for the design of compounds that can act as uncompetitive inhibitors of a CE. These compounds can bind to all, or a portion of, an accessory binding site of a CE that is already binding its ligand and can, therefore, be more potent and less non-specific than known competitive inhibitors that compete only for the CE ligand binding cavity. Similarly, non-competitive inhibitors that bind to and inhibit CE activity, whether or not it is bound to another chemical entity, can be designed using the CE structure coordinates of this invention.

A second design approach is to probe a CE crystal with molecules comprising a variety of different chemical entities to determine optimal sites for interaction between candidate CE modulators and the polypeptide. For example, high resolution X-ray diffraction data collected from crystals saturated with solvent allows the determination of the site where each type of solvent molecule adheres. Small molecules that bind tightly to those sites can then be designed, synthesized and tested for their CE modulator activity.

Once a computationally-designed ligand is synthesized using the methods of the present invention or other methods known to those of skill in the art, assays can be employed to establish the efficacy of the ligand as a modulator of CE activity. After such assays, the ligands can be further refined by generating intact CE crystals with a ligand bound to the CE. The structure of the ligand can then be further refined using the chemical modification methods described herein and known to those of skill in the art, in order to improve the modulation activity or the binding affinity of the ligand. This process can lead to second generation ligands with improved properties.

Optionally, interactions of a CE polypeptide are targeted. Suitable assays for screening that can be employed, mutatis mutandis in the present invention, are described in published PCT international applications WO 00/037077 and WO 00/025134, incorporated herein by reference in their entirety.

V.A.3. Methods of Designing CE Modulator Compounds

The design of candidate substances, also referred to as “compounds” or “candidate compounds”, that enhance or inhibit CE-mediated activity according to the present invention generally involves consideration of two factors. First, the compound must be capable of physically and structurally associating with a CE. Non-covalent molecular interactions important in the association of a CE with its substrate include hydrogen bonding, van der Waals interactions and hydrophobic interactions.

Second, the compound must be able to assume a conformation that allows it to associate with a CE. Although certain portions of the compound might not directly participate in this association with a CE, those portions can still influence the overall conformation of the molecule. This, in turn, can have a significant impact on potency. Such conformational requirements include the overall three-dimensional structure and orientation of the chemical entity or compound in relation to all or a portion of the binding site, e.g., a ligand binding cavity or an accessory binding site of a CE, or the spacing between functional groups of a compound comprising several chemical entities that directly interact with a CE.

The potential modulatory or binding effect of a chemical compound on a CE can be analyzed prior to its actual synthesis and testing by employing computer modeling techniques that employ the coordinates of a crystalline CE polypeptide of the present invention. If the theoretical structure of the given compound suggests insufficient interaction and association between it and a CE, synthesis and testing of the compound is obviated. However, if computer modeling indicates a strong interaction, the molecule can then be synthesized and tested for its ability to bind and modulate the activity of a CE. In this manner, synthesis of unproductive or inoperative compounds can be avoided.

A modulatory or other binding compound of a CE polypeptide can be computationally evaluated and designed via a series of steps in which chemical entities or fragments are screened and selected for their ability to associate with the individual binding sites or other areas of a crystalline CE polypeptide of the present invention.

One of several methods can be used to screen chemical entities or fragments for their ability to associate with a CE and, more particularly, with the individual binding sites of a CE, such as ligand binding cavity or an accessory binding site. This process can begin by visual inspection of, for example, a ligand binding cavity on a computer screen based on the CE atomic coordinates in Tables 3, 6 and 7. Selected fragments or chemical entities can then be positioned in a variety of orientations, or docked, within an individual binding site of a CE as defined herein above. Docking can be accomplished using software programs such as those available under the tradenames QUANTA™ (Molecular Simulations Inc., San Diego, Calif.) and SYBYL™ (Tripos, Inc., St. Louis, Mo.), followed by energy minimization and molecular dynamics with standard molecular mechanics forcefields, such as CHARM (Brooks et al., (1983) J. Comp. Chem., 8: 132) and AMBER 5 (Case et al., (1997), AMBER 5, University of California, San Francisco; Pearlman et al., (1995) Comput. Phys. Commun. 91: 1-41).

Specialized computer programs can also assist in the process of selecting fragments or chemical entities. These include:

1. GRID™ program, version 17 (Goodford, (1985) J. Med. Chem. 28: 849-57), which is available from Molecular Discovery Ltd., Oxford, UK;

2. MCSS™ program (Miranker & Karplus, (1991) Proteins 11: 29-34), which is available from Accelrys, San Diego, Calif.;

3. AUTODOCK™ 3.0 program (Goodsell & Olsen, (1990) Proteins 8: 195-202), which is available from the Scripps Research Institute, La Jolla, Calif.;

4. DOCK™ 4.0 program (Kuntz et al., (1992) J. Mol. Biol. 161: 269-88), which is available from the University of California, San Francisco, Calif.;

5. FLEX-X™ program (Rarey et al., (1996) J. Comput. Aid. Mol. Des. 10:41-54), which is available from Tripos, Inc., St. Louis, Mo.;

6. MVP program (Lambert, (1997) in Practical Application of Computer-Aided Drug Design, (Charifson, ed.) Marcel-Dekker, New York, pp. 243-303); and

7. LUDI™ program (Bohm, (1992) J. Comput. Aid. Mol. Des., 6: 61-78), which is available from Accelrys, San Diego, Calif.

Once suitable chemical entities or fragments have been selected, they can be assembled into a single compound or modulator. Assembly can proceed by visual inspection of the relationship of the fragments to each other on the three-dimensional image displayed on a computer screen in relation to the structure coordinates of a CE. Manual model building using software such as QUANTA™ or SYBYL™ typically follows.

Useful programs to aid one of ordinary skill in the art in connecting the individual chemical entities or fragments include:

1. CAVEAT™ program (Bartlett et al., (1989) Special Pub., Royal Chem. Soc. 78: 182-96), which is available from the University of California, Berkeley, Calif.;

2. 3D Database systems, such as MACCS-3D™ system program, which is available from MDL Information Systems, San Leandro, Calif. This area is reviewed in Martin, (1992) J. Med. Chem. 35: 2145-54; and

3. HOOK™ program (Eisen et al., (1994). Proteins 19: 199-221), which is available from Accelrys, San Diego, Calif.

Instead of proceeding to build a CE modulator in a step-wise fashion one fragment or chemical entity at a time as described above, modulatory or other binding compounds can be designed as a whole or de novo using the structural coordinates of a crystalline CE polypeptide of the present invention and either an empty binding site or optionally including some portion(s) of a known modulator(s). Applicable methods can employ the following software programs:

1. LUDI™ program (Bohm, (1992) J. Comput. Aid. Mol. Des., 6: 61-78), which is available from Accelrys, San Diego, Calif.;

2. LEGEND™ program (Nishibata & Itai, (1991) Tetrahedron 47: 8985); and

3. LEAPFROG™, which is available from Tripos Associates, St. Louis, Mo.

Other molecular modeling techniques can also be employed in accordance with this invention. See, e.g., Cohen et al., (1990) J. Med. Chem. 33: 883-94. See also, Navia & Murcko, (1992) Curr. Opin. Struc. Biol. 2: 202-10; U.S. Pat. No. 6,008,033, herein incorporated by reference.

Once a compound has been designed or selected by the above methods, the efficiency with which that compound can bind to a CE can be tested and optimized by computational evaluation. By way of particular example, a compound that has been designed or selected to function as a CE modulator can also traverse a volume not overlapping that occupied by the binding site when it is bound to its native ligand. Additionally, an effective CE modulator can demonstrate a relatively small difference in energy between its bound and free states (i.e., a small deformation energy of binding). Thus, the most efficient CE modulators can be designed with a deformation energy of binding of not greater than, for example, about 10 kcal/mole, or, for example, not greater than 7 kcal/mole. It is possible for CE modulators to interact with the polypeptide in more than one conformation that is similar in overall binding energy. In those cases, the deformation energy of binding is taken to be the difference between the energy of the free compound and the average energy of the conformations observed when the modulator binds to the polypeptide.

A compound designed, known or suspected to bind to a CE polypeptide can be further computationally optimized so that in its bound state it would lack repulsive electrostatic interaction with the target polypeptide. Such non-complementary (e.g., electrostatic) interactions include repulsive charge-charge, dipole-dipole and charge-dipole interactions. For example, the sum of all electrostatic interactions between the modulator and the polypeptide when the modulator is bound to a CE can make a neutral or favorable contribution to the enthalpy of binding.

Specific computer software is available in the art to evaluate compound deformation energy and electrostatic interaction. Examples of programs designed for such uses include:

1. Gaussian 98™, which is available from Gaussian, Inc., Pittsburgh, Pa.;

2. AMBER™ program, version 6.0, which is available from the University of California at San Francisco;

3. QUANTA™ program, which is available from Accelrys, San Diego, Calif.;

4. CHARM® program, which is available from Accelrys, San Diego, Calif.; and

4. INSIGHT II® program, which is available from Accelrys, San Diego, Calif.

These programs can be implemented using a suitable computer system. Other hardware systems and software packages will be apparent to those skilled in the art after review of the disclosure of the present invention presented herein.

Once a CE modulating compound has been optimally selected or designed, as described above, substitutions can then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds can then be analyzed for efficiency of fit to a CE binding site using the same computer-based approaches described in detail above.

V.B. Distinguishing Between CE Isoforms and Orthologs

The present invention discloses the ability to generate new synthetic ligands to distinguish between CE isoforms and orthologs. As described herein, computer-designed ligands can be generated that distinguish between binding isoforms and orthologs, thereby allowing the generation of species specific, tissue specific or function specific ligands. The atomic structural coordinates disclosed in the present invention reveal structural details unique to a mammalian CE in general and a rabbit CE (e.g., rCE) or a human CE (e.g., hCE1) in particular. These structural details can be exploited when a novel ligand is designed using the methods of the present invention or other ligand design methods known in the art. The structural features that differentiate a human CE from a rabbit CE, for example, and one isoform from another can be targeted in ligand design. Thus, for example, a ligand can be designed that will recognize a particular CE isoform or ortholog, while not interacting with other CE isoforms or orthologs, or even with moieties having similar structural features. Prior to the disclosure of the present invention, a detailed understanding of the differences between CE orthologs and/or isoforms, and the ability to target a particular CE isoform or ortholog, was unattainable.

V.C. Method of Screening for Chemical and Biological Modulators of the Biological Activity of CE

A candidate substance can be further analyzed in a screening assay of the present invention to confirm an ability to modulate the biological activity of a CE polypeptide. In one embodiment, such a candidate compound can have utility in the treatment of disorders and conditions associated with the biological activity of a CE polypeptide as noted above, including, but not limited to, CE-based drug-drug interactions, CE-based drug resistance, individualized treatment of disease due to polymorphisms, cancer and other cancer-related disorders, activation of prodrugs, cholesteryl ester formation and hydrolysis, sex hormone maturation, lung surfactant generation, treatment of Alzheimer's Disease, fatty acid ethyl ester formation associated with alcohol abuse, and malaria invasion into human liver.

In a cell-free system, the method comprises the steps of establishing a control system comprising a CE polypeptide and a ligand which is capable of binding to the polypeptide; establishing a test system comprising a CE polypeptide, the ligand, and a candidate compound; and determining whether the candidate compound modulates the activity of the polypeptide by comparison of the test and control systems. A representative ligand comprises CPT-11, tacrine, homatropine or other small molecule, and in this embodiment, the biological activity or property screened includes binding affinity.

In another embodiment of the invention, a crystalline form of a CE polypeptide or a catalytic or immunogenic fragment or oligopeptide thereof, can be used to screen libraries of compounds in any of a variety of drug screening techniques. The fragment employed in such a screening can be affixed to a solid support. The formation of binding complexes, between a CE polypeptide and the agent being tested, can be detected. In one embodiment, the CE polypeptide has an amino acid sequence of SEQ ID NO: 2 or SEQ ID NO 4.

Another technique for drug screening that is facilitated by the present invention provides for high throughput screening of compounds having suitable binding affinity to the protein of interest as described in published PCT application WO 84/03564, herein incorporated by reference. In this method, as applied to a polypeptide of the present invention, large numbers of different small test compounds are synthesized on a solid substrate, such as plastic pins or some other surface. The test compounds are reacted with the polypeptide, or fragments thereof. Bound polypeptide is then detected by methods well known to those of skill in the art. The polypeptide can also be placed directly onto plates for use in the aforementioned drug screening techniques.

In yet another embodiment, a method of screening for a modulator of a CE polypeptide or a CE polypeptide comprises: providing a library of test samples; contacting a CE polypeptide with each test sample; detecting an interaction between a test sample and a CE polypeptide or a CE polypeptide; identifying a test sample that interacts with a CE polypeptide or a CE polypeptide; and isolating a test sample that interacts with a CE polypeptide.

In each of the foregoing embodiments, an interaction can be detected spectrophotometrically, radiologically or immunologically. An interaction between a CE polypeptide and a test sample can also be quantified using methodology known to those of skill in the art. In another embodiment, the CE polypeptide is in crystalline form.

In accordance with the present invention there is also provided a rapid and high throughput screening method that relies on the methods described above. This screening method comprises separately contacting each of a plurality of substantially identical samples with a CE polypeptide and detecting a resulting binding complex. In such a screening method the plurality of samples can comprise, for example, more than about 10⁴ samples, or for example more more than about 5×10⁴ samples.

V.D. Method of Identifying Compounds that Inhibit Ligand Binding

Using the disclosed crystal structures and ligand orientations, disclosed for the first time herein, it is possible to design test compounds that inhibit binding of ligands normally bound by a CE polypeptide.

In one aspect of the present invention, an assay method for identifying a compound that inhibits binding of a ligand to a CE polypeptide is disclosed. A known ligand of CE can be used in the assay method as the ligand against which the inhibition by a test compound is gauged. CPT-11, 4PP, tacrine and homatropine, for example, can be ligands in the assay method. The method comprises (a) incubating a CE polypeptide with a ligand in the presence of a test inhibitor compound; (b) determining an amount of ligand that is bound to the CE polypeptide, wherein decreased binding of ligand to the CE polypeptide in the presence of the test inhibitor compound relative to binding in the absence of the test inhibitor compound is indicative of inhibition; and (c) identifying the test compound as an inhibitor of ligand binding if decreased ligand binding is observed.

In another aspect of the present invention, the disclosed assay method can be used in the structural refinement of candidate CE inhibitors. For example, multiple rounds of optimization can be followed by gradual structural changes in a strategy of inhibitor design. A strategy such as this is made possible by the disclosure of the atomic structural coordinates of a mammalian CE and the disclosure of the orientation of a ligand of CE, for example CPT-11, 4PP, tacrine or homatropine.

V.E. Design of CE Isoform and Ortholog Modulators

The rabbit CE (e.g. rCE) or human CE (e.g. hCE1) crystal structures of the present invention can be used to generate modulators of other CE isoforms or orthologs, such as human or mouse CE. Analysis of the disclosed crystal structure can provide a guide for designing modulators of CE isoforms or orthologs. For purposes of explanation, the development of a mouse CE modulator will be considered herein below. It will be apparent to those of skill in the art, and explicitly noted here, that the following discussion will be applicable mutatis mutandis to CE isoforms and other CE orthologs.

Absent the crystal structure of the present invention, researchers would be required to design mouse CE modulators de novo. The present invention, however, addresses this problem by providing insights into the binding cavity of a rabbit CE (e.g., rCE) and/or a human CE (e.g., hCE1), which can be extended, due to significant structural similarity with other CE isoforms and orthologs, to the binding cavity of, for example, mouse CE. An evaluation of the binding cavity of a rabbit CE (e.g., rCE) and/or a human CE (e.g., hCE1) indicates that a potential mouse CE modulator would meet a broad set of general criteria. Broadly, it can be stated that, based on the crystal structures of a rabbit CE (e.g., rCE) and/or a human CE (e.g., hCE1), a potent mouse CE ligand would require several general features including: (a) the ability to interact with/in a hydrophobic binding cavity; and (b) the ability to adopt a conformation that is complementary to the shape of the binding cavity.

Using the discerned structural similarities and differences between CE isoforms and orthologs, as represented and predicted based on the crystal structure of the present invention and homology models, a human CE modulator can be designed. For example, based on an evaluation of a homology model of mouse CE, which is derived from the disclosed rabbit CE (e.g., rCE) and/or a human CE (e.g., hCE1) crystal structures, it is expected that a potent ligand would need similar characteristics as listed above for a compound recognized by rabbit CE (e.g., rCE) and/or a human CE (e.g., hCE1). Additional modifications can be included, based on the disclosed structure, which are predicted to further define a modulator specific for mouse CE over other orthologs. Thus, the disclosed crystal structures of rabbit CE (e.g., rCE) and/or a human CE (e.g., hCE1) can be useful when designing modulators of mouse CE and other orthologs and isoforms.

### VI. Design, Preparation and Structural Analysis of CE Mutants and Structural Equivalents

The present invention provides for the generation of CE mutants, and the ability to solve the crystal structures of those that crystallize. More particularly, through the provision of the three-dimensional structure of rabbit CE (e.g., rCE) and/or a human CE (e.g., hCE1), desirable sites for mutation can be identified, based on analysis of the three-dimensional rabbit CE (e.g., rCE) and/or a human CE (e.g., hCE1) structure coordinates provided herein.

The structure coordinates of rabbit CE (e.g., rCE) and/or a human CE (e.g., hCE1) provided in accordance with the present invention also facilitate the identification of related proteins or enzymes analogous to rabbit CE (e.g., rCE) and/or a human CE (e.g., hCE1) in function, structure or both, (for example, a mouse CE), which can lead to novel therapeutic modes for treating or preventing a range of disease states, including those described above.

VI.A. Sterically Similar Compounds

A further aspect of the present invention is that sterically similar compounds can be formulated to mimic the key portions of a rabbit CE (e.g., rCE) and/or a human CE (e.g., hCE1) structure. Such compounds are functional equivalents. The generation of a structural functional equivalent can be achieved by the techniques of modeling and chemical design known to those of skill in the art and described herein. Modeling and chemical design of CE structural equivalents can be based on the structure coordinates of a crystalline rabbit CE (e.g., rCE) and/or a human CE (e.g., hCE1) polypeptide of the present invention. It will be understood that all such sterically similar constructs fall within the scope of the present invention.

VI.B. CE Polypeptides

The generation of chimeric CE polypeptides is also an aspect of the present invention. Such a chimeric polypeptide can comprise a CE polypeptide or a portion of a CE, which is fused to a candidate polypeptide or a suitable region of the candidate polypeptide, for example a CE expressed in mouse or other species. Throughout the present disclosure it is intended that the term “mutant” encompass not only mutants of a CE polypeptide but chimeric proteins generated using a CE as well. It is thus intended that the following discussion of mutant CEs apply mutatis mutandis to chimeric CE polypeptides and to structural equivalents thereof.

In accordance with the present invention, a mutation can be directed to a particular site or combination of sites of a wild-type CE. For example, an accessory binding site or the binding cavity can be chosen for mutagenesis. Similarly, a residue having a location on, at or near the surface of the polypeptide can be replaced, resulting in an altered surface charge of one or more charge units, as compared to the wild-type CE. Alternatively, an amino acid residue in a CE can be chosen for replacement based on its hydrophilic or hydrophobic characteristics.

Such mutants can be characterized by any one of several different properties as compared with the wild-type CE. For example, such mutants can have an altered surface charge of one or more charge units, or can have an increase in overall stability. Other mutants can have altered substrate specificity in comparison with, or a higher specific activity than, a wild-type CE.

CE mutants of the present invention can be generated in a number of ways. For example, the wild-type sequence of a CE can be mutated at those sites identified using this invention as desirable for mutation, by means of oligonucleotide-directed mutagenesis or other conventional methods, such as deletion. Alternatively, mutants of a CE can be generated by the site-specific replacement of a particular amino acid with an unnaturally occurring amino acid. In addition, CE mutants can be generated through replacement of an amino acid residue, for example, a particular cysteine or methionine residue, with selenocysteine or selenomethionine. This can be achieved by growing a host organism capable of expressing either the wild-type or mutant polypeptide on a growth medium depleted of either natural cysteine or methionine (or both) but enriched in selenocysteine or selenomethionine (or both).

A mutation can be introduced into a DNA sequence coding for a CE using synthetic oligonucleotides. These oligonucleotides contain nucleotide sequences flanking the desired mutation sites. A mutation can be generated in the full-length DNA sequence of a CE or in any sequence coding for polypeptide fragments of a CE.

According to the present invention, a mutated CE DNA sequence produced by the methods described above, or any alternative methods known in the art, can be expressed using an expression vector. An expression vector, as is well known to those of skill in the art, typically includes elements that permit autonomous replication in a host cell independent of the host genome, and one or more phenotypic markers for selection purposes. Either prior to or after insertion of the DNA sequences surrounding the desired CE mutant coding sequence, an expression vector also will include control sequences encoding a promoter, operator, ribosome binding site, translation initiation signal, and, optionally, a repressor gene or various activator genes and a signal for termination. In some embodiments, where secretion of the produced mutant is desired, nucleotides encoding a “signal sequence” can be inserted prior to a CE mutant coding sequence. For expression under the direction of the control sequences, a desired DNA sequence must be operatively linked to the control sequences; that is, the sequence must have an appropriate start signal in front of the DNA sequence encoding the CE mutant, and the correct reading frame to permit expression of that sequence under the control of the control sequences and production of the desired product encoded by that CE sequence must be maintained.

Any of a wide variety of well-known available expression vectors can be useful in the expression of a mutated CE coding sequence of this invention. These expression vectors can be used in the techniques disclosed in the Laboratory Examples and can include, for example, vectors comprising segments of chromosomal, non-chromosomal and synthetic DNA sequences, such as various known derivatives of SV40, known bacterial plasmids, e.g., plasmids from E. coli including col E1, pCR1, pBR322, pMB9 and their derivatives, wider host range plasmids, e.g., RP4, phage DNAs, e.g., the numerous derivatives of phage λ, e.g., NM 989, and other DNA phages, e.g., M13 and filamentous single stranded DNA phages, yeast plasmids and vectors derived from combinations of plasmids and phage DNAs, such as plasmids which have been modified to employ phage DNA or other expression control sequences. In one embodiment of this invention, the E. coli vector pRSETA, including a T7-based expression system, is employed.

In addition, any of a wide variety of expression control sequences—equences that control the expression of a DNA sequence when operatively linked to it—can be used in these vectors to express the mutated DNA sequences according to this invention. Such useful expression control sequences, include, for example, the early and late promoters of SV40 for animal cells, the lac system, the trp system the TAC or TRC system, the major operator and promoter regions of phage λ, the control regions of fd coat protein, all for E. coli the promoter for 3-phosphoglycerate kinase or other glycolytic enzymes, the promoters of acid phosphatase, e.g., Pho5, the promoters of the yeast α-mating factors for yeast, and other sequences known to control the expression of genes of prokaryotic or eukaryotic cells or their viruses, and various combinations thereof.

A wide variety of hosts are also useful for producing mutated CE polypeptides according to this invention. These hosts include, for example, bacteria, such as E. coli, Bacillus and Streptomyces, fungi, such as yeasts, and animal cells, such as CHO and COS-1 cells, plant cells, insect cells, such as Sf9 and Sf21 cells, and transgenic host cells.

It should be understood that not all expression vectors and expression systems function in the same way to express mutated DNA sequences of this invention, and to produce modified CE polypeptides or CE mutants. Neither do all hosts function equally well with the same expression system. One of ordinary skill in the art can, however, make a selection among these vectors, expression control sequences and hosts without undue experimentation and without departing from the scope of this invention. For example, an important consideration in selecting a vector will be the ability of the vector to replicate in a given host. The copy number of the vector, the ability to control that copy number, and the expression of any other proteins encoded by the vector, such as antibiotic markers, should also be considered.

When selecting an expression control sequence, a variety of factors should also be considered. These include, for example, the relative strength of the system, its controllability and its compatibility with the DNA sequence encoding a modified CE polypeptide of this invention, with particular regard to the formation of potential secondary and tertiary structures.

Hosts should be selected by consideration of their compatibility with the chosen vector, the toxicity of a modified CE to them, their ability to express mature products, their ability to fold proteins correctly, their fermentation requirements, the ease of purification of a modified CE and safety. Within these parameters, one of skill in the art can select various vector/expression control system/host combinations that will produce useful amounts of a mutant CE. A mutant CE produced in these systems can be purified by a variety of conventional steps and strategies, including those used to purify the wild-type CE.

Once a CE mutation(s) has been generated in the desired location, such as a ligand binding site, the mutants can be tested for any one of several properties of interest. For example, mutants can be screened for an altered charge at physiological pH. This can be determined by measuring the mutant CE isoelectric point (pl) and comparing the observed value with that of the wild-type parent. Isoelectric point can be measured by gel-electrophoresis according to the method of Wellner (Wellner, (1971) Anal. Chem. 43: 597). A mutant CE polypeptide containing a replacement amino acid located at the surface of the enzyme, as provided by the structural information of this invention, can lead to an altered surface charge and an altered pl.

VI.C. Generation of an Engineered CE or CE Mutant

In another aspect of the present invention, a unique CE polypeptide can be generated. Such a mutant can facilitate purification and/or can facilitate the study of the ligand binding abilities of a CE polypeptide.

As used herein, the terms “engineered CE” and “CE mutant” refer to polypeptides having amino acid sequences that contain at least one mutation in the wild-type sequence. The terms also refer to CE polypeptides which are capable of exerting a biological effect in that they comprise all or a part of the amino acid sequence of an engineered CE mutant polypeptide of the present invention, or cross-react with antibodies raised against an engineered CE mutant polypeptide, or retain all or some or an enhanced degree of the biological activity of the engineered CE mutant amino acid sequence or protein. Such biological activity can include ligand binding.

The terms “engineered CE” and “CE mutant” also includes analogs of a CE mutant polypeptide. By “analog” is intended that a DNA or polypeptide sequence can contain alterations relative to the sequences disclosed herein, yet retain all or some or an enhanced degree of the biological activity of those sequences. Analogs can be derived from genomic nucleotide sequences or from other organisms, or can be created synthetically. Those of skill in the art will appreciate that other analogs, as yet undisclosed or undiscovered, can be used to design and/or construct CE mutant analogs. There is no need for a CE mutant polypeptide to comprise all or substantially all of the amino acid sequence of SEQ ID NOs: 2 or 4. Shorter or longer sequences are anticipated to be of use in the invention; shorter sequences are herein referred to as “segments”. Thus, the terms “engineered CE” and “CE mutant” also includes fusion, chimeric or recombinant engineered CE or CE mutant polypeptides and proteins comprising sequences of the present invention. Methods of preparing such proteins are disclosed herein above and are known in the art.

VI.D. Sequence Similarity and Identity

As used herein, the term “substantially similar” means that a particular sequence varies from nucleic acid sequence of SEQ ID NOs: 1 or 3 or the amino acid sequence of SEQ ID NOs: 2 or 4 by one or more deletions, substitutions, or additions, the net effect of which is to retain at least some of biological activity of the natural gene, gene product, or sequence. Such sequences include “mutant” or “polymorphic” sequences, or sequences in which the biological activity and/or the physical properties are altered to some degree but retains at least some or an enhanced degree of the original biological activity and/or physical properties. In determining nucleic acid sequences, all subject nucleic acid sequences capable of encoding substantially similar amino acid sequences are considered to be substantially similar to a reference nucleic acid sequence, regardless of differences in codon sequences or substitution of equivalent amino acids to create biologically functional equivalents.

VI.D.1. Sequences that are Substantially Identical to a CE Mutant Sequence of the Present Invention

Nucleic acids that are substantially identical to a nucleic acid sequence of a CE mutant of the present invention, e.g. allelic variants, genetically altered versions of the gene, etc., bind to a CE mutant sequence under stringent hybridization conditions. By using probes, particularly labeled probes of DNA sequences, one can isolate homologous or related genes. The source of homologous genes can be any species, e.g. primate species; rodents, such as rats and mice, canines, felines, bovines, equines, yeast, nematodes, etc.

Between mammalian species, e.g. human and mouse, homologs have substantial sequence similarity, i.e. at least 75% sequence identity between nucleotide sequences. Sequence similarity is calculated based on a reference sequence, which can be a subset of a larger sequence, such as a conserved motif, coding region, flanking region, etc. A reference sequence can be, for example, at least about 18 nucleotides (nt) long, or in another example, at least about 30 nucleotides long, and can extend to the complete sequence that is being compared. Algorithms for sequence analysis are known in the art, such as BLAST, described in Altschul et al., (1990) J. Mol. Biol. 215: 403-10.

Percent identity or percent similarity of a DNA or peptide sequence can be determined, for example, by comparing sequence information using the GAP computer program, available from the University of Wisconsin Geneticist Computer Group. The GAP program utilizes the alignment method of Needleman et al., (1970) J. Mol. Biol. 48: 443, as revised by Smith et al., (1981) Adv. Appl. Math. 2:482. Briefly, the GAP program defines similarity as the number of aligned symbols (i.e., nucleotides or amino acids) that are similar, divided by the total number of symbols in the shorter of the two sequences. Parameters for the GAP program can be, for example, the default parameters, which do not impose a penalty for end gaps. See, e.g., Schwartz et al., eds., (1979), Atlas of Protein Sequence and Structure, National Biomedical Research Foundation, pp. 357-358, and Gribskov et al., (1986) Nucl. Acids. Res. 14: 6745.

The term “similarity” is contrasted with the term “identity”. Similarity is defined as above; “identity”, however, means a nucleic acid or amino acid sequence having the same amino acid at the same relative position in a given family member of a gene family. Homology and similarity are generally viewed as broader terms than the term identity. Biochemically similar amino acids, for example leucine/isoleucine or glutamate/aspartate, can be present at the same position—these are not identical per se, but are biochemically “similar.” As disclosed herein, these are referred to as conservative differences or conservative substitutions. This differs from a conservative mutation at the DNA level, which changes the nucleotide sequence without making a change in the encoded amino acid, e.g. TCC to TCA, both of which encode serine.

As used herein, DNA analog sequences are “substantially identical” to specific DNA sequences disclosed herein if: (a) the DNA analog sequence is derived from coding regions of the nucleic acid sequence shown in SEQ ID NOs: 1 and 3; or (b) the DNA analog sequence is capable of hybridization with DNA sequences of (a) under stringent conditions and which encode a biologically active CE gene product; or (c) the DNA sequences are degenerate as a result of alternative genetic code to the DNA analog sequences defined in (a) and/or (b). Substantially identical analog proteins and nucleic acids will have, for example, between about 70% and 80%, or about 81% to about 90% or about 91% and 99% sequence identity with the corresponding sequence of the native protein or nucleic acid. Sequences having lesser degrees of identity but comparable biological activity are considered to be equivalents.

As used herein, “stringent conditions” means conditions of high stringency, for example 6×SSC, 0.2% polyvinylpyrrolidone, 0.2% Ficoll, 0.2% bovine serum albumin, 0.1% sodium dodecyl sulfate, 100 μ/ml salmon sperm DNA and 15% formamide at 68° C. For the purposes of specifying additional conditions of high stringency, conditions can comprise, for example, a salt concentration of about 200 mM and temperature of about 45° C. One example of such stringent conditions is hybridization at 4×SSC, at 65° C., followed by a washing in 0.1×SSC at 65° C. for one hour. Another example stringent hybridization scheme uses 50% formamide, 4×SSC at 42° C.

In contrast, nucleic acids having sequence similarity are detected by hybridization under lower stringency conditions. Thus, sequence identity can be determined by hybridization under lower stringency conditions, for example, at 50° C. or higher and 0.1×SSC (9 mM NaCl/0.9 mM sodium citrate) and the sequences will remain bound when subjected to washing at 55° C. in 1×SSC.

VI.D.2. Complementarity and Hybridization to a CE Mutant Sequence

As used herein, the term “complementary sequences” means nucleic acid sequences that are base-paired according to the standard Watson-Crick complementarity rules. The present invention also encompasses the use of nucleotide segments that are complementary to the sequences of the present invention.

Hybridization can also be used for assessing complementary sequences and/or isolating complementary nucleotide sequences. As discussed above, nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of about 30° C., typically in excess of about 37° C., and or temperatures in excess of about 45° C. Stringent salt conditions will ordinarily be less than about 1,000 mM, less than about 500 mM, or less than about 200 mM. However, the combination of parameters is much more important than the measure of any single parameter. See, e.g., Wetmur & Davidson, (1968) J. Mol. Biol. 31: 349-70. Determining appropriate hybridization conditions to identify and/or isolate sequences containing high levels of homology is well known in the art. See, e.g., Sambrook et al., (1989) Molecular Cloning: A Laboratory Manual, Cold Spring Harbor, N.Y.

VI.D.3. Functional Equivalents of a CE Mutant Nucleic Acid Sequence of the Present Invention

As used herein, the term “functionally equivalent codon” is used to refer to codons that encode the same amino acid, such as the ACG and AGU codons for serine. CE-encoding nucleic acid sequences comprising SEQ ID NOs: 1 and 3, which have functionally equivalent codons, are covered by the present invention. Thus, when referring to the sequence example presented in SEQ ID NOs: 1 and 3, applicants contemplate substitution of functionally equivalent codons into the sequence examples of SEQ ID NOs: 1 and 3. Thus, applicants are in possession of amino acid and nucleic acids sequences which include such substitutions but which are not set forth herein in their entirety for convenience.

It will also be understood by those of skill in the art that amino acid and nucleic acid sequences can include additional residues, such as additional N-or C-terminal amino acids or 5′ or 3′ nucleic acid sequences, and yet still be essentially as set forth in one of the sequences disclosed herein, so long as the sequence retains biological protein activity where polypeptide expression is concerned. The addition of terminal sequences particularly applies to nucleic acid sequences which can, for example, include various non-coding sequences flanking either of the 5′ or 3′ portions of the coding region or can include various internal sequences, i.e., introns, which are known to occur within genes.

VI.D.4. Biological Equivalents

The present invention envisions and includes biological equivalents of a CE mutant polypeptide of the present invention. The term “biological equivalent” refers to proteins having amino acid sequences which are substantially identical to the amino acid sequence of a CE mutant of the present invention and which are capable of exerting a biological effect in that they are capable of binding DNA moieties or cross-reacting with anti-CE mutant antibodies raised against a mutant CE polypeptide of the present invention.

For example, certain amino acids can be substituted for other amino acids in a protein structure without appreciable loss of interactive capacity with, for example, structures in the nucleus of a cell. Since it is the interactive capacity and nature of a protein that defines that protein's biological functional activity, certain amino acid sequence substitutions can be made in a protein sequence (or the nucleic acid sequence encoding it) to obtain a protein with the same, enhanced, or antagonistic properties. Such properties can be achieved by interaction with the normal targets of the protein, but this need not be the case, and the biological activity of the invention is not limited to a particular mechanism of action. It is thus in accordance with the present invention that various changes can be made in the amino acid sequence of a CE mutant polypeptide of the present invention or its underlying nucleic acid sequence without appreciable loss of biological utility or activity.

Biologically equivalent polypeptides, as used herein, are polypeptides in which certain, but not most or all, of the amino acids can be substituted. Thus, when referring to the sequence examples presented in SEQ ID NOs: 1 and 3, applicants envision substitution of codons that encode biologically equivalent amino acids, as described herein, into the sequence examples of SEQ ID NOs: 2 and 4, respectively. Thus, applicants are in possession of amino acid and nucleic acids sequences which include such substitutions but which are not set forth herein in their entirety for convenience.

Alternatively, functionally equivalent proteins or peptides can be created via the application of recombinant DNA technology, in which changes in the protein structure can be engineered, based on considerations of the properties of the amino acids being exchanged, e.g. substitution of Ile for Leu. Changes designed by man can be introduced through the application of site-directed mutagenesis techniques, e.g., to introduce improvements to the antigenicity of the protein or to test a mutant CE polypeptide of the present invention in order to modulate DNA-binding, lipid-binding or other activity, at the molecular level.

Amino acid substitutions, such as those which might be employed in modifying a mutant CE polypeptide of the present invention are generally, but not necessarily, based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all of similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents. Other biologically functionally equivalent changes will be appreciated by those of ordinary skill in the art. It is implicit in the above discussion, however, that one of skill in the art can appreciate that a radical, rather than a conservative substitution is warranted in a given situation. Non-conservative substitutions in mutant CE polypeptides of the present invention are also an aspect of the present invention.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, (1982), J. Mol. Biol. 157: 105-132, incorporated herein by reference). It is known that certain amino acids can be substituted for other amino acids having a similar hydropathic index or score and still retain a similar biological activity. In making changes based upon the hydropathic index, the substitution of amino acids whose hydropathic indices are within, for example, ±2, ±1, or ±0.5 of the original value can also be employed.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within, for example, ±2, ±1 or within ±0.5 of the original value can be employed.

While discussion has focused on functionally equivalent polypeptides arising from amino acid changes, it will be appreciated that these changes can be effected by alteration of the encoding DNA, taking into consideration also that the genetic code is degenerate and that two or more codons can code for the same amino acid.

Thus, it will also be understood that this invention is not limited to the particular amino acid and nucleic acid sequences of SEQ ID NOs: 1-4. Recombinant vectors and isolated DNA segments can therefore variously include a mutant CE polypeptide-encoding region itself, include coding regions bearing selected alterations or modifications in the basic coding region, or include larger polypeptides which nevertheless comprise a mutant CE polypeptide-encoding region or can encode biologically functional equivalent proteins or polypeptides which have variant amino acid sequences. Biological activity of a mutant CE polypeptide can be determined, for example, by ligand-binding assays known to those of ordinary skill in the art.

The nucleic acid segments of the present invention, regardless of the length of the coding sequence itself, can be combined with other DNA sequences, such as promoters, enhancers, polyadenylation signals, additional restriction enzyme sites, multiple cloning sites, other coding segments, and the like, such that their overall length can vary considerably. It is therefore contemplated that a nucleic acid fragment of almost any length can be employed, with the total length being a reflection of, for example, the ease of preparation and use in the intended recombinant DNA protocol. For example, nucleic acid fragments can be prepared which include a short stretch complementary to a nucleic acid sequence set forth in SEQ ID NOs: 1 and 3, such as about 10 nucleotides, and which are up to 10,000 or 5,000 base pairs in length. DNA segments with total lengths of about 4,000, 3,000, 2,000, 1,000, 500, 200, 100, and about 50 base pairs in length can also be employed.

The DNA segments of the present invention encompass biologically functional equivalents of mutant CE polypeptides. Such sequences can rise as a consequence of codon redundancy and functional equivalency that are known to occur naturally within nucleic acid sequences and the proteins thus encoded. Alternatively, functionally equivalent proteins or polypeptides can be created via the application of recombinant DNA technology, in which changes in the protein structure can be engineered, based on considerations of the properties of the amino acids being exchanged. Changes can be introduced through the application of site-directed mutagenesis techniques, e.g., to introduce improvements to the antigenicity of the protein or to test variants of a mutant CE of the present invention in order to examine a degree of small molecule binding activity, or other activity at the molecular level. Various site-directed mutagenesis techniques are known to those of ordinary skill in the art and can be employed in the present invention.

The invention further encompasses fusion proteins and peptides wherein a mutant CE coding region of the present invention is aligned within the same expression unit with other proteins or peptides having desired functions, such as for purification or immunodetection purposes.

Recombinant vectors form important further aspects of the present invention. Particularly useful vectors are those in which the coding portion of the DNA segment is positioned under the control of a promoter. The promoter can be that naturally associated with a CE gene, as can be obtained by isolating the 5′ non-coding sequences located upstream of the coding segment or exon, for example, using recombinant cloning and/or PCR technology and/or other methods known in the art, in conjunction with the compositions disclosed herein.

In other embodiments, certain advantages will be gained by positioning the coding DNA segment under the control of a recombinant, or heterologous, promoter. As used herein, a recombinant or heterologous promoter is a promoter that is not normally associated with a CE gene in its natural environment. Such promoters can include promoters isolated from bacterial, viral, eukaryotic, or mammalian cells. Naturally, it will be important to employ a promoter that effectively directs the expression of the DNA segment in the cell type chosen for expression. The use of promoter and cell type combinations for protein expression is generally known to those of skill in the art of molecular biology (see, e.g., Sambrook et al., (1989) Molecular Cloning: A Laboratory Manual, Cold Spring Harbor Laboratory, New York, incorporated herein by reference). The promoters employed can be constitutive or inducible and can be used under the appropriate conditions to direct high level expression of the introduced DNA segment, such as is advantageous in the large-scale production of recombinant proteins or peptides. One example promoter system contemplated for use in high-level expression is a T7 promoter-based system.

VI.E. Uses of CE Mutants

The CE mutants disclosed herein have a variety of applications, including in the screening compounds for CE binding using the cell-free reporter gene assay methods disclosed herein above, and using whole animal models. The CE mutants can also be used in cell-free, cell-based and whole animal assay methods for bioavailability of compounds and for toxicology analysis. Additionally, CE mutants can be employed in crystallizations, screening for changes in ligand activation and screening for species-specific changes in ligand activation, both with and without ligand.

### VII. The Role of the Three-Dimensional Structure of the CE in Solving Additional CE Crystals

Because polypeptides can crystallize in more than one crystal form, the structural coordinates of a mammalian CE (e.g., a rCE or hCE1), or portions thereof, as provided in one embodiment of the present invention, are particularly useful in solving the structure of other crystal forms of rabbit CE (e.g., rCE), a human CE (e.g., hCE1) and/or the crystalline forms of other mammalian and non-mammalian CEs. Indeed, the rCE structure disclosed herein was employed as a template in the molecular replacement solutions of the hCE1-tacrine and hCE1-homatropine structures that form aspects of the present invention. The coordinates provided in the present invention can also be used to solve the structure of CE mutants (such as those described above), CE co-complexes, or of the crystalline form of any other protein with significant amino acid sequence homology to any functional region of CE (see Table 1, for a non-limiting list of example sequences).

One method that can be employed for the purpose of solving additional CE crystal structures is molecular replacement. See generally, The Molecular Replacement Method, Rossmann, (ed.) Gordon & Breach, New York (1972). In the molecular replacement method, the unknown crystal structure, whether it is another crystal form of a CE, or a CE polypeptide complexed with another compound (a “co-complex”), or the crystal of some other protein with significant amino acid sequence homology to any functional region of the a CE, can be determined using the structure coordinates provided in Tables 3, 6 and 7. This method provides an accurate structural form for the unknown crystal more quickly and efficiently than attempting to determine such information ab initio.

In addition, in accordance with this invention, CE mutants can be crystallized in complex with known modulators. The crystal structures of a series of such complexes can then be solved by molecular replacement and compared with that of wild-type CE. Potential sites for modification within the various binding sites of the enzyme can thus be identified. This information provides an additional tool for determining the most efficient binding interactions, for example, increased hydrophobic interactions, between the CE and a chemical entity or compound.

All of the complexes referred to in the present disclosure can be studied using X-ray diffraction techniques (see, e.g., Blundell & Johnson (1985) Method.Enzymol. 114A & 115B, (Wyckoff et al., eds.), Academic Press) and can be refined using computer software, such as the X-PLOR™ program (Brünger, (1992) X-PLOR, Version 3.1. A System for X-ray Crystallography and NMR, Yale University Press, New Haven, Conn.; X-PLOR is available from Accelrys, San Diego, Calif.). This information can thus be used to optimize known classes of CE modulators, and more importantly, to design and synthesize novel classes of CE modulators.

### LABORATORY EXAMPLES

The Laboratory Examples have been included to illustrate various representative modes of the invention. Certain aspects of the Laboratory Examples are described in terms of techniques and procedures found or contemplated by the present co-inventors to work well in the practice of the invention. These Laboratory Examples are exemplified through the use of standard laboratory practices of the inventors. In light of the present disclosure and the general level of skill in the art, those of skill will appreciate that the Laboratory Examples are intended to be exemplary only and that numerous changes, modifications and alterations can be employed without departing from the spirit and scope of the invention.

### Methods Employed in Laboratory Examples 1-4

Crystallization and Crystal Handling

A 62 kDa truncated form of rCE (SEQ ID NO.: 2) lacking six C-terminal amino acids was used. The enzyme was expressed using a baculovirus expression system in Spodoptera frugiperda Sf21 cells with the expressed enzyme secreted into the culture media. rCE was purified by preparative isoelectric focusing and size-exclusion chromatography (BIO-GEL® P-100, Bio-Rad Laboratories, Hercules, Calif., United States of America) from protein-free culture media, in accordance with techniques disclosed by Morton & Potter (Morton & Potter, (2000) Mol. Biotechnol. 16: 193-202). Purified rCE was concentrated to 3 mg ml⁻¹ in 50 mM HEPES, pH 7.4, and crystallized in the presence of 4PP at 1,000-fold molar excess relative to protein concentration. Crystals (300×300×200 μm³) were grown by sitting drop vapor diffusion at 22° C. in 10% (w/v) PEG 3350, 0.1M Li₂SO₄, 0.1M citrate, pH 5.5, and 5% (v/v) glycerol for 5-14 days, and were cryo-protected in 30% (v/v) glycerol plus mother liquor before flash cooling in liquid nitrogen.

Structure Determination and Refinement

Diffraction data were collected at Stanford Synchrotron Radiation Laboratory (SSRL) beamline 9-1, and processed and reduced using DENZO and SCALEPACK. Otwinowski & Minor, (1993) Data Collection and Processing, Daresbury Laboratories, Warrington, Chesire, United Kingdom. Crystals were of space group R32, and crystal density calculations (Matthews, (1968) J. Mol. Biol. 33: 491-497) (Vₘ=2.78 Å³ Da⁻¹) indicated one molecule in the asymmetric unit. The structure of rCE was determined by molecular replacement using the structure of acetylcholinesterase (AcChE; PDB entry 2ACE) (Harel, (1993) et al. Proc. Natl. Acad. Sci. U.S.A. 90: 9031-9035) from Torpedo californica as a search model (31% sequence identity). Nonidentical side chains and four short inserts (2-7 residues in length) were trimmed before rotation and translation function searches in AmoRe (Navaza & Saludjian, (1997) Methods Enzymol. 276A, 581-594). The structure was refined using torsion angle dynamics in CNS (Brünger et al., (1998). Acta Crystallogr. D 54: 905-921) with the maximum likelihood function target, and included an overall anisotropic B-factor and a bulk solvent correction. Before refinement, 7% of the observed data were set aside for cross-validation using R_{free}. Brünger et al., (1998) Acta Crystallogr. D 54: 905-921. Manual adjustments and rebuilding were performed using O (Jones et al., (1991) Acta Crystallogr. A 47: 110-119) and σ_{A}-weighted (Read, (1986) Acta Crystallogr. A 42: 140-149) electron density maps. At the later stages of refinement, the N-linked glycans and 388 waters were added.

The electron density adjacent to the Asn 389 carbohydrate group was carefully analyzed using σ_{A}-weighted difference-density and simulated annealing omit maps. Positioning the following molecules into this density was attempted: citrate, HEPES, glycerol and a covalently linked fucose carbohydrate group. Only a 4PP molecule, the product of CPT-11 activation, fit and refined well into this density. Without 1,000-fold molar excess 4PP, crystals were highly mosaic and showed relatively poor diffraction. The final rCE structure, evaluated by PROCHECK (Laskowski et al., (1993) J. Appl. Crystallogr. 26: 283-291), shows good geometry (Table 2), with 85% of the protein residues lying in most favored regions of the Ramachandran plot and 15% lying in additionally allowed regions. Only a single protein residue, the catalytic Ser 221, lies in a generously allowed region that is conserved in several esterase structures. Harel et al., (1993) Proc. Natl. Sci. U.S.A. 90: 9031-9035; Sussman et al., (1993) Chem. Biol. Interact. 87: 187-197; Kryger et al., (1999) Structure Fold Des. 7: 297-307. Molecular graphic figures were created with MolScript (Kraulis, (1991) Appl. Crystallogr. 24: 946-950), BobScript (Esnouf, (1999) Acta. Crystallogr. D 55: 938-940) and Raster3D (Merritt & Bacon, (1997) Methods Enzymol. 277: 505-524). Closely related structures were identified by DALI (Holm & Sander, (1997) Nucleic Acids Res. 25: 231-234).

Thermal Denaturation Studies

Experiments were conducted by monitoring rCE denaturation in an Applied Photophysics PISTAR-180™ CD spectropolarimeter (Applied Photophysics, Surrey, United Kingdom). Deglycosylated rCE was generated by an 18-hour treatment with 0.25 μM PNGase F (Hampton Research, Laguna Niguel, Calif., United States of America) at 37° C., which cleaves the complete high-mannose glycosyl group. Removal of the carbohydrate chains was confirmed by SDS-PAGE. Wild type protein was also heated to 37° C. for 18 hours before CD experiments. Wild type or deglycosylated rCE (0.15 mg ml⁻¹ (2.5 μM); in 10 mM phosphate buffer, pH 7.0, and 1 mM fresh beta-mercaptoethanol (βME) to eliminate the stabilizing effect of disulfide linkages) was treated with no 4PP or with 0.016 mM, 0.16 mM, 1.6 mM or 10 mM 4PP. The temperature was increased from 20 to 98° C. while monitoring the ellipticity at 222 nm. Plots of fraction denatured versus temperature were produced by defining the upper and lower temperature baselines as 0 and 100%, respectively.

Coordinates

The coordinates of the rCE structure have been deposited with the Protein Data Bank (RCSB Protein Id No. 1K4Y), and are also presented herein in Table 3.

### Laboratory Example 1

rCE Comprises Three Domains

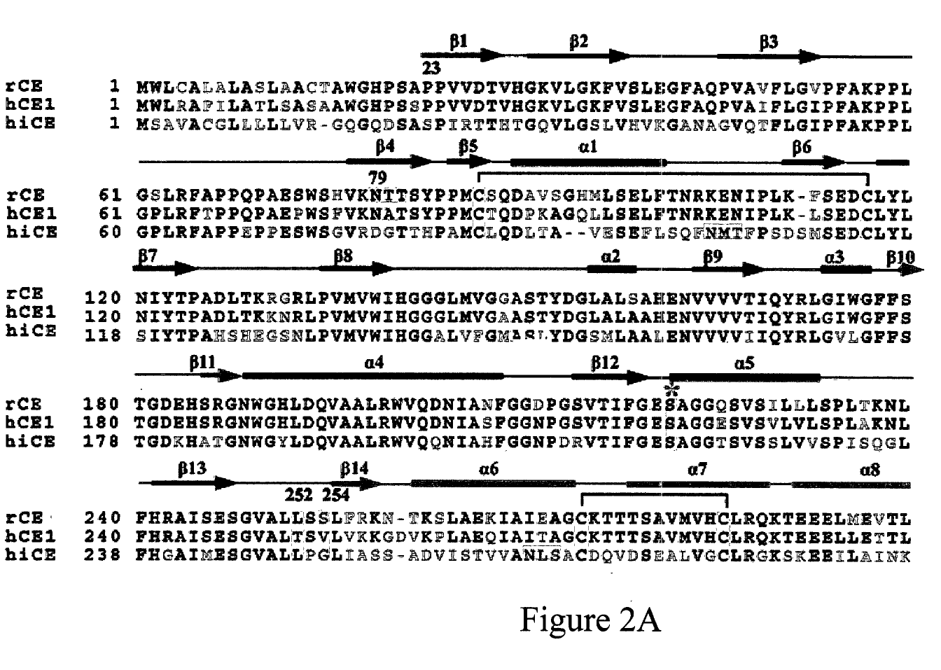

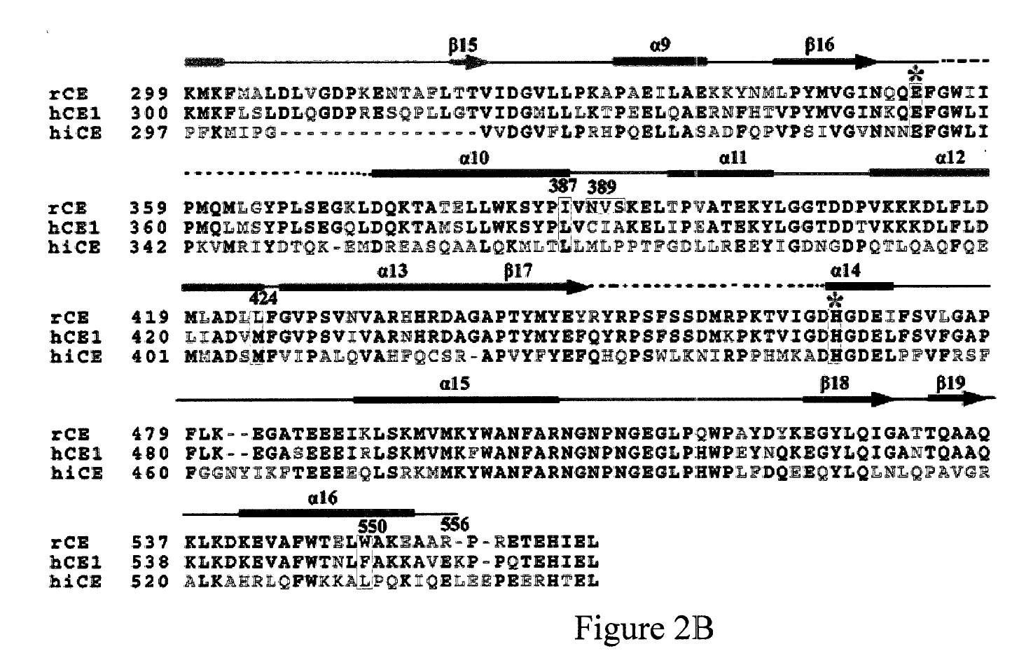

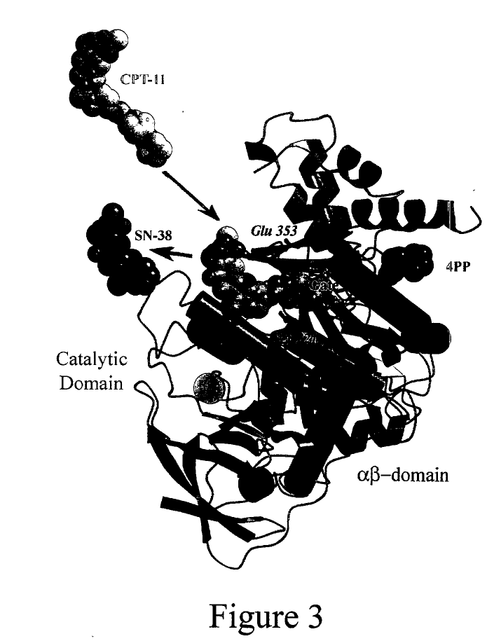

The structure of rCE was determined by molecular replacement using the structure of Torpedo californica acetylcholinesterase (tAcChE) as a search model (Harel et al., (1993) Proc. Natl. Acad. Sci. U.S.A. 90: 9031-9035) and was refined to 2.5 Å resolution. Residues 23-354, 371-449 and 467-556 of the 565-amino acid long rCE enzyme were positioned, along with 99 carbohydrate atoms, the 24-atom 4-piperidino-piperidine (4PP) group and 388 water molecules. Two 16-amino acid loops (355-370 and 450-466) are disordered and not present in the final model (FIG. 3). The enzyme comprises a catalytic domain (blue in FIG. 3), an αβ domain (green in FIG. 3) and a regulatory domain (red in FIG. 3). Within the catalytic domain, the enzyme shows the common α/β hydrolase fold, comprising a central antiparallel β-sheet surrounded by α-helices (FIG. 3). The secondary structural elements within this catalytic domain (α4, α5, α13, α15, β7-β9 and β12-β13) are the most conserved in sequence with the human CEs (FIGS. 2A and 2B). The αβ domain (α6-8, β10-11 and β14-15) lies adjacent to both the catalytic and regulatory domains. The regulatory domain is α-helical (α10-12 and α16) and includes the C-terminal helix of the enzyme. rCE is stabilized by two conserved disulfide linkages, one between Cys 87 and Cys 116, and one between Cys 273 and Cys 284.

Although the secondary structural elements within the catalytic domain of rCE are similar in structure to tAcChE (r.m.s. deviation of 0.5 Å over 99 equivalent Cα positions), other regions within rCE deviate significantly from tAcChE. For example, the region between residues 90 and 102 is a helix (α1) in rCE and shifted 12 Å relative to the equivalent 15-amino acid loop (termed the Ω-loop, residues 72-86) in tAcChE. The αβ domain of rCE also exhibits a 5 Å rigid-body shift in position relative to the equivalent region in tAcChE and contains loops shifted by >10 Å—for example, rCE residues 300-317 versus tAcChE residues 278-291.

### Laboratory Example 2

Flexibility at the Active Site

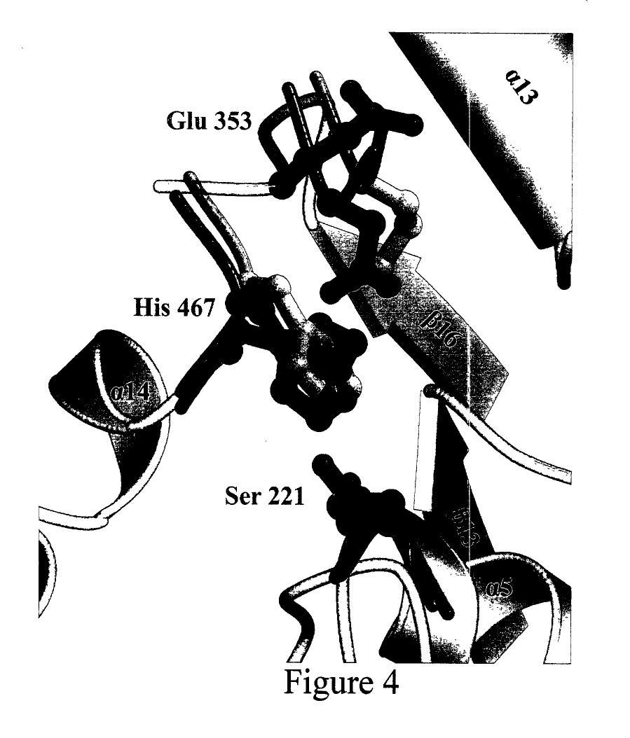

Ser 221, Glu 353 and His 467, conserved residues in the human CEs, form a rCE catalytic triad (FIG. 4). The catalytic Ser 221 is located at the bottom of a ˜25 Å deep active site cleft, approximately in the center of the molecule. The other members of the catalytic triad of rCE, Glu 353 and His 467, are located adjacent to the two disordered loops in the structure (355-370 and 450-466). The catalytic Glu 353 of rCE is rotated away from the active site relative, to orientations observed in other esterases. Glu 353 and His 467 lie adjacent to regions of structural disorder in rCE. The substrate-binding region of rCE is formed by upper and lower jaws that surround the active site gorge, similar to that observed for the acetylcholinesterases (AcChEs). See Harel et al., (1993) Proc. Natl. Acad. Sci. U.S.A. 90: 9031-9035; Sussman et al., (1993) Chem. Biol. Interact 87: 187-197; Kryger & Silman, (1999) Structure Fold Des. 7: 297-307. A cluster of four α-helices (α10-α13) form the upper jaw, and the lower jaw is composed of two α-helices (α1 and α8) and the loop between β15 and α8 (FIGS. 3 and 4). While it is not the inventors' desire to be bound by any particular theory of operation, it appears that the two 16-residue loops not present in the rCE structure are expected to partially close over this entrance to the active site region of the enzyme. The structural flexibility of these loops could play a role in the catalytic cycle of the enzyme.

The positions of the rCE catalytic residues were compared with those of related esterases with known structure (Table 1). FIG. 4 depicts the active site of rCE (green) superimposed on that of two esterases closely related in structure: triacylglycerol hydrolase (PBD entry 1THG; gold) and cholesterol esterase (2BCE; magenta). When triacylglycerol hydrolase (Schrag & Cygler, (1993) J. Mol. Biol. 230: 575-591) (1.8 Å r.m.s. deviation over 544 Cα positions) and cholesterol esterase (Chen et al., (1998) Biochem. 37: 5101-5117) (2.2 Å r.m.s. deviation over 532 Cα positions) are superimposed onto rCE, the Cα backbone around the catalytic sites line up well (FIG. 4). However, the positions of the rCE catalytic residues deviate from those in triacylglycerol hydrolase and cholesterol esterase. In particular, the rCE catalytic Glu 353 residue is rotated ˜3 Å away from the equivalent negatively charged residues in these enzymes. Although it is not the inventors' desire to be bound to any theory of operation, because Glu 353 and His 467 are located immediately adjacent to the two regions of disorder in rCE (355-370 and 450-466), these observations suggest that the flexibility of the surface loops of rCE can impact the positions of active site residues, affecting the catalytic function of the enzyme. In particular, these observations suggest that the active site might not form until the substrate is bound productively within the catalytic gorge.

### Laboratory Example 3

Asn-Linked Glycosylation Sites

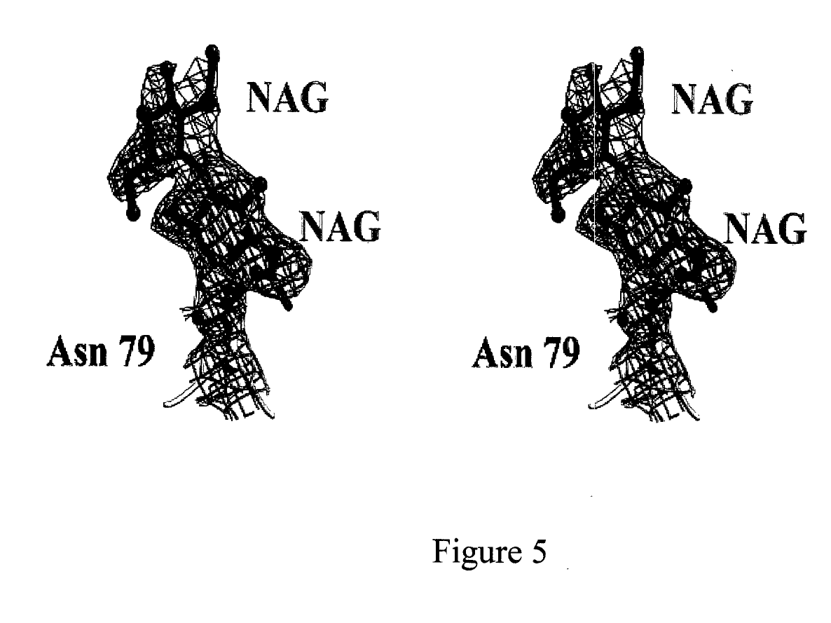

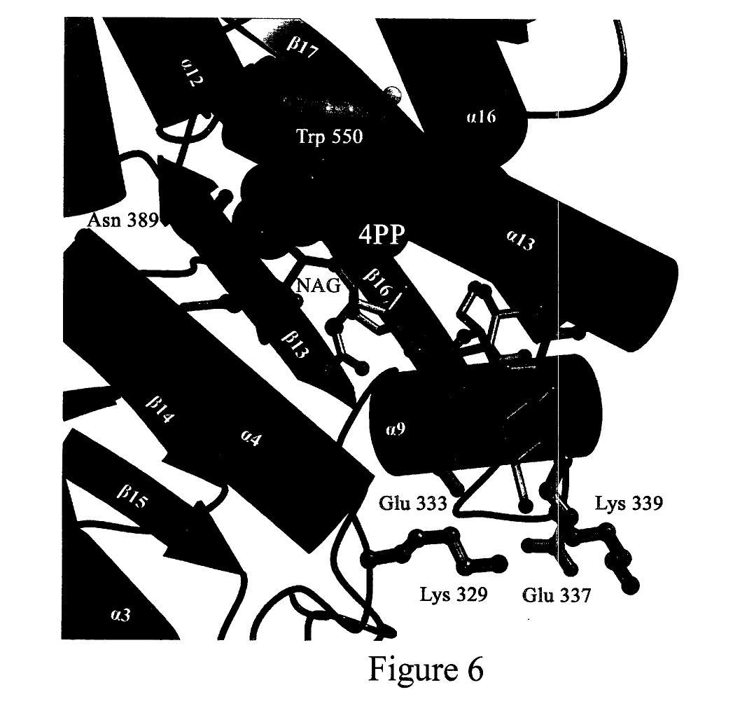

Posttranslational oligosaccharide modifications assist with the localization, folding, solubility and circulatory half-life of many eukaryotic proteins. Helenius & Aebi, (2001) Science 291, 2364-2369. Two sites of N-linked glycosylation were identified in rCE at Asn residues 79 and 389 (FIGS. 2A and 2B). FIG. 5 is a stereo view of a composite simulated-annealing omit map (cyan; contoured at 1.0 σ) and the final σ_{A}-weighted (Read, (1986) Acta Crystallog. A 42:140-149) 2Fₒ-F_{c} map (magenta; contoured at 1.0 σ) around the Asn 79 glycosylation site in rCE (both maps at 2.5 Å resolution). As FIG. 5 indicates, Asn 79 is modified by two N-acetylglucosamine (NAG) groups. At Asn 389 in rCE, a longer carbohydrate chain composed of the sequence NAG-NAG-MAN-2MAN (MAN, for mannose) (colored cyan in FIG. 6) was traced. This carbohydrate moiety appears to link the central region of the protein to the C-terminal helix (α16) and bridge the gap between the Asn side chain and an adjacent patch of charged residues. By sequence analysis, hCE1 appears to maintain the glycosylation site at Asn 79 but not at residue 389. In contrast, hCE1 contains glycosylation sites at two positions (residues 103 and 267) distinct from those observed in rCE (FIGS. 2A and 2B).

### Laboratory Example 4

4PP Binding on rCE Surface

Persistent electron density was observed adjacent to the Asn 389 high-mannose glycosylation site in rCE. This region showed maximum peak heights of 3.8 σ in 2.5 Å resolution difference density maps calculated before building waters or the glycosyl groups. Several candidate molecules were positioned and refined into this density. Only a 4PP molecule, a product of CPT-11 activation, fit and refined well. 4PP binds between the first NAG of the Asn 389 glycosylation site and the Trp 550 side chain of the C-terminal helix in the rCE structure (FIG. 6).

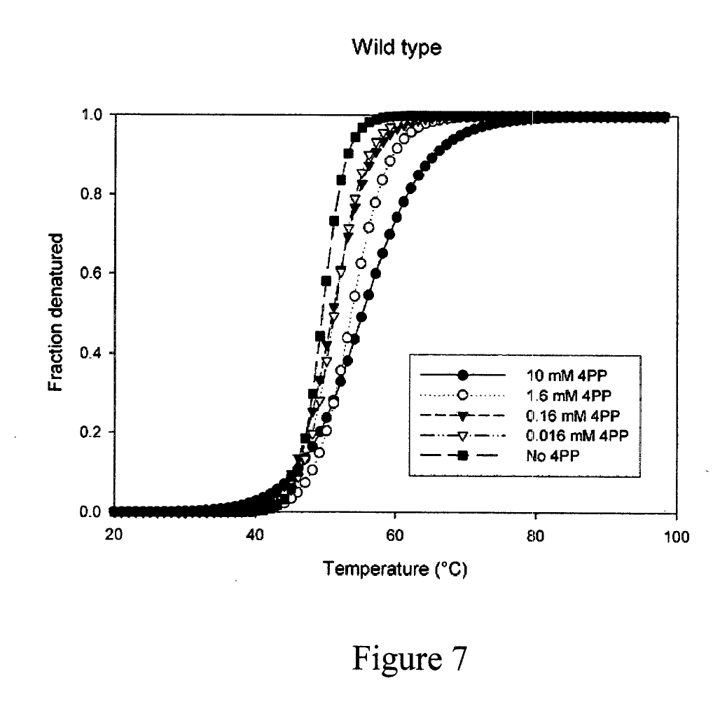

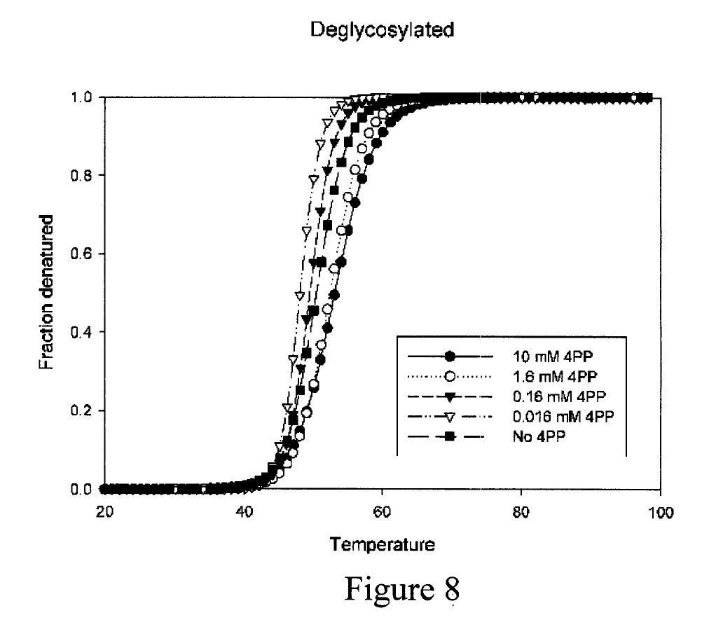

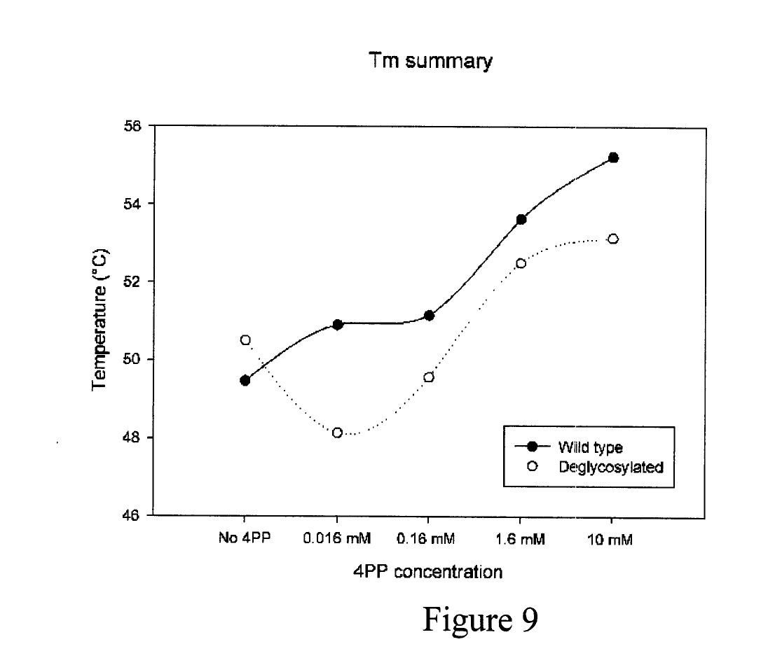

To confirm the significance of the 4PP bound to the surface of rCE, thermal denaturation studies were performed using CD on both wild type and deglycosylated rCE. Deglycosylated rCE was generated using peptide-N4-(acetyl-β-glucosaminyl)-asparagine amidase (PNGase F), which cleaves the complete high-mannose carbohydrate chain and leaves an unmodified Asn residue. In the presence of fresh reducing agent (1 mM β-mercaptoethanol (βME)), thermal denaturation of wild type and deglycosylated rCE was monitored alone and in the presence of 10-(0.016 mM), 100-(0.16 mM), 1,000-(1.6 mM) and 6,000-fold (10 mM) molar excess 4PP (FIGS. 7-9). The melting temperature (Tₘ) of wild type rCE is increased to 51° C. with 10- to 100-fold molar excess of 4PP, whereas the presence of higher concentrations of 4PP added additional stability to the enzyme (Tₘ=54-55° C.) (FIG. 7). Using deglycosylated rCE, stabilization occurs only with high concentrations of 4PP, whereas lower concentrations destabilize the enzyme (FIG. 8). An examination of the rCE Tₘ by 4PP concentration (FIG. 9) suggests that there are two classes of binding sites for 4PP on wild type rCE: specific binding that is occupied by 10- to 100-fold excess 4PP and nonspecific binding that becomes occupied only at higher 4PP concentrations. The deglycosylated form of the protein, in contrast, seems to allow only nonspecific binding, because high concentrations of 4PP are required for stabilization. 4PP is present at 1,000-fold molar excess in the crystallization conditions using wild type rCE. While it is not applicants' desire to be bound by any theory of operation, because 4PP bound only at the Asn 389 glycosylation site, it is proposed that this is the specific 4PP-binding site on the enzyme. Nonspecific binding of 4PP could occur at the active site of the enzyme or elsewhere on the molecule.

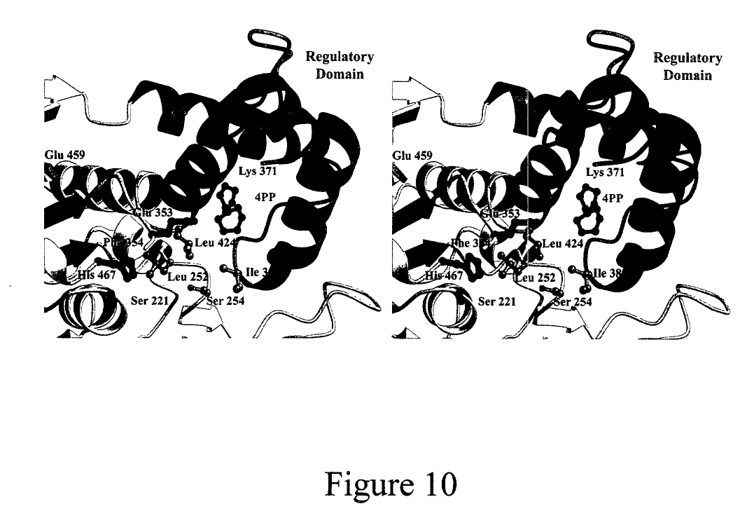

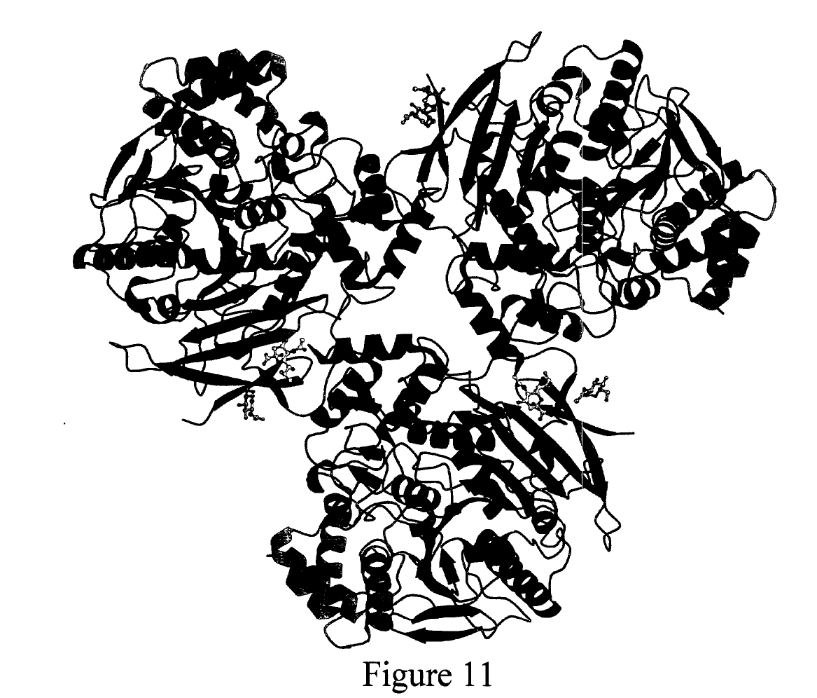

While it is not the inventors' desire to be bound by any theory of operation, the crystallographic observation of 4PP binding to the Asn 389 glycosylation site suggests that a novel exit pore exists in rCE to facilitate the release of small products from the active site of the enzyme. Such a pore would be similar to the “back door” exit proposed for acetylcholinesterases. Gilson et al., (1994) Science 263: 1276-1278; Bartolucci et al., (1999) Biochem. 38: 5714-5719. Four residues were identified that could “gate” a product exit pore in rCE: Leu 252, Ser 254, Ile 387 and Leu 424. These residues line the deepest region of the substrate-binding pocket (35 Å from the surface of the enzyme) and form a thin wall that separates the active site from the 4PP binding site, as depicted in FIGS. 10 and 11. Thus, they could gate the release of products from the rCE catalytic site. In FIG. 10, the regulatory domain is depicted in red and comprises helices α9, α10, α11 and α14. Gate residues are Leu 252, Ser 254, Ile 387 and Leu 424 and are depicted in cyan. The residues that mark the beginning and end of the disordered regions of the structure (Phe 354, Lys 371, Glu 459 and His 467) are also labeled. The active site is in green and bound 4PP molecule is in magenta.

### Laboratory Example 5

Proposed Mechanism of CPT-11 Activation

A “back door” has long been postulated to facilitate the release of small products from the active site of AcChE. Gilson et al., (1994) Science 263: 1276-1278. The direct crystallographic visualization of the product 4PP bound to the surface of rCE led to a consideration that rCE could also use an alternative product exit pore akin to the AcChE back door. Such considerations are supported by the proximity of this surfacebinding site to the catalytic region of the enzyme (15 Å) and by the observation that four gate residues (Leu 252, Ser 254, Ile 387 and Leu 424) (FIGS. 10 and 11) separate 4PP from the active site. However, the 4PP binding site observed in the rCE structure is located ˜180° away from the product exit pore proposed for AcChE. Thus, the putative product exit pore in rCE is referred to as the “side door”.

Two additional lines of evidence further support this proposed side door product exit site. First, 4PP facilitates the generation of stable crystals of rCE. The presence of other compounds similar to SN-38, the other product of CPT-11 activation, or the standard esterase assay product o-nitrophenol do not yield useful crystals. Second, removal of the high-mannose glycosylation groups eliminates the stabilizing effects of low concentrations of 4PP (FIGS. 7-9), indicating that the specific binding of 4PP is dependent on carbohydrate.

rCE appears to use two groups of residues to dictate substrate selectivity. First, amino acids located on the walls of the active site gorge form the alcohol site and interact with the SN-38 portion of CPT-11. Second, deep within the substrate-binding cavity, the four gate residues form the acyl site and interact with the 4PP moiety. Recent mutagenesis studies of rat lung CE (rLCE) and rat hepatic neutral cytosolic cholesteryl ester hydrolase (rhncCEH) indicate that the equivalent residues in these enzymes are important for substrate selectivity. Wallace et al., (2001) J. Biol. Chem. 276: 33165-33174. rLCE and rhncCEH differ in sequence by only four amino acids. One such residue, Met 423 in rLCE and Ile 423 in rhncCEH, is equivalent to Leu 424 in rCE. An M4231 mutation in rLCE changes the substrate performance at rLCE to that of rhncCEH, which prefers more hydrophobic substrates. A similar situation might exist within rCE (with Leu 424) and hCE1 (with Met 424), suggesting that the rCE gate residues might be critical for substrate selectivity.

The dipiperidino region of CPT-11, which forms the 4PP leaving group when cleaved, fits well into the deepest portion of the binding pocket, adjacent to the putative gate residues (FIGS. 3 and 10). Although it is not the inventors' desire to be bound by any theory of operation, it is proposed that after CPT-11 is cleaved by rCE, the alcohol product (SN-38) exits out of the active site gorge, but the acyl product (4PP) exits through the side door past the two pairs of residues gating this pore: Leu 252 and Ser 254, and Ile 387 and Leu 424 (FIGS. 3 and 10).

FIG. 6 is a schematic depicting an orientation of the “side door” binding site for 4PP in rCE. In FIG. 6, the acyl product of CPT-11 activation (4PP) is shown in magenta. In this figure, the acyl product is stacked in between the indole ring side chain of Trp 550 (yellow) and the proximal NAG (cyan) attached to Asn 389. The rCE catalytic domain is shown in blue; the rCE αβ domain is shown in green; and the rCE regulatory domain is shown in red.

Continuing, FIG. 3 illustrates a proposed mechanism for the activation of CPT-11 by rCE. CPT-11 (orange) enters from the top of the catalytic gorge and fits well into the active site (catalytic Ser 221 and Glue 353 in green). After cleavage, the alcohol product (SN-38; magenta) leaves via the catalytic gorge, while the acyl product (4PP; magenta) moves past the gate residues (cyan) and docks adjacent to the regulatory domain (red) on the surface of the molecule. The regulatory domain then rotates back down to close transiently over the 4PP at the side door, which causes the active site gorge to open and the loops covering the active site to become disordered. The structure presented in FIG. 3 has a resolution of 2.5 Å. After new substrate binds at the active site, the regulatory domain rotates back over the active site to interact with substrate, allowing the 4PP group to leave the surface binding site.

Thus, the first crystallographic evidence of product bound adjacent to a putative esterase secondary exit channel is presented herein. These results advance understanding of esterase function and the ability of mammalian carboxylesterases to act on a wide variety of substrates. In addition, these results facilitate the design of novel CPT-11 analogs or engineered forms of rCE for use in cancer chemotherapy.

### Laboratory Example 6

hCE1 Crystallization and Crystal Handling

A 62 kDa C-terminally-truncated form of human carboxylesterase 1 (hCE1; SEQ ID NO: 4) that lacks six C-terminal amino acids, allowing secretion of the expressed enzyme (baculovirus in Spodoptera frugiperda Sf21 cells) into the culture media, was employed. hCE1 was purified by preparative isoelectric focusing and size exclusion chromatography (BIO-GEL® P-100, Bio-Rad, Laboratories, Hercules, Calif.) from protein-free culture media. Purified hCE1 was concentrated to 5 mg ml⁻¹ in 20 mM HEPES pH 7.4, and crystallized in the presence of either 10 mM tacrine or 100 mM homatropine. Crystals were grown by sitting drop vapor diffusion at 22° C. in 8% PEG-3350, 0.4 M Li₂SO₄, 0.1 M LiCl, 0.1 M NaCl, 0.1 M citrate pH 5.5, 5% glycerol in 14-28 days, and were cryo-protected in 15% sucrose plus mother liquor prior to flash cooling in liquid nitrogen. Crystal and data statistics obtained from the crystallized LBD of human CE in complex of either tacrine or homatropine are presented in Table 5, while coordinate data for the hCE1-homatropine structure is presented in Table 6 and coordinate data for the hCE1-tacrine structure is presented in Table 7.

### Laboratory Example 7

hCE1 Structure Determination and Refinement

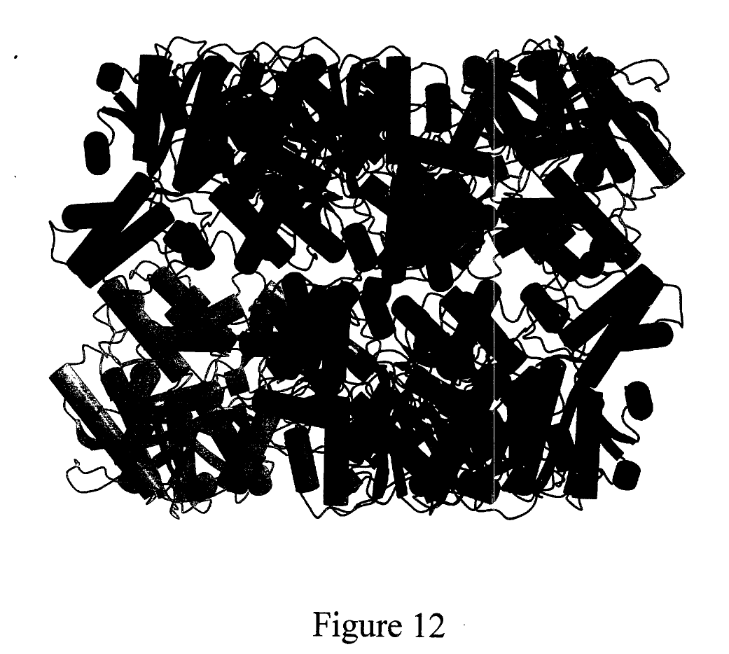

Diffraction data were collected at Stanford Synchrotron Radiation Laboratory (SSRL) beamline 9-1, and were processed and reduced using MOSFILM (Leslie, (1992), Joint CCP4+ESF-EAMCB Newsletter on Protein Crystallography, No. 26.). Crystals were of space group P2₁, and contained six molecules in the asymmetric unit for both the tacrine and homatropine complexes. See FIG. 12, in which each hCE1 is depicted in a different color. The structures of hCE1 were determined by molecular replacement using the structure of rabbit carboxylesterase (rCE; RCSB Protein ID No. 1K4Y; Bencharit et al., (2002) Nat.Struct.Biol. 9: 337), also an aspect of the present invention, as a search model (81% sequence identity). Non-identical side chains were trimmed prior to rotation and translation function searches in AmoRe (Navaza & Saludjian, (1997) Methods Enzymol. 276A: 581-594). The structures were refined using torsion angle dynamics in CNS with the maximum likelihood function target, and included an overall anisotropic B-factor and a bulk solvent correction. Non-crystallographic symmetry restraints were used at early stages of refinement, and then removed such that six independent molecules were refined for both the tacrine and homatropine complexes. 10% of the observed data were set aside for cross-validation using free-R prior to refinement. Manual adjustments and rebuilding were performed using the program O (Jones et al., (1991) Acta Crystallogr. A 47: 110-119) and σ_{A}-weighted electron density maps (Read, (1986) Acta Crystallogr. A 42: 140-149). At the later stages of refinement the N-linked glycans and waters were added. Tacrine was placed in multiple orientations at the active site of hCE1 using standard and simulated annealing difference maps, as well as computational results from BLOB. Homatropine was placed at the active site of hCE1 using standard and simulated annealing difference maps, and at the surface site using similar maps and guidance from BLOB computational results. The final hCE1 structures (FIGS. 11-14) were evaluated by PROCHECK (Laskowski et al., (1993) J. Appl. Crystallogr. 26: 283-291), and exhibit good geometry. As depicted in FIG. 11, hCE1 appears to adopt a trimeric configuration. In FIG. 11, each hCE is depicted in green, red and blue. The an region is depicted in green, the regulatory domain is depicted in red and the catalytic domain is depicted in blue.

### References

The references here as well as all references cited in the specification are incorporated herein by reference to the extent that they supplement, explain, provide a background for or teach methodology, techniques and/or compositions employed herein.

Albert & Gledhill, (1945) J. Chem. Soc. Ind. 64: 169T

Altschul et al., (1990) J. Mol. Biol. 215: 403-10

Ausubel et al., (1989) Current Protocols in Molecular Biology, Greene Publishing Associates and Wiley Interscience, New York

Bartlett et al., (1989) Special Pub., Royal Chem. Soc. 78: 182-96

Bartolucci et al., (1999) Biochem. 38: 5714-5719

Bencharit et al., (2002) Nat.Struct.Biol. 9: 337

Blundell & Johnson, (1985) Method.Enzymol. 114A & 115B, (Wyckoff et al., eds.), Academic Press)

Blundell et al., (1976) Protein Crystallography, Academic Press

Bodor & Buchwald, (2000) Med. Res. Rev. 20: 58-101

Bohm, (1992) J. Comput. Aid. Mol. Des., 6: 61-78

Brooks et al., (1983) J. Comp. Chem., 8: 132

Brünger et al., (1998) Acta Crystallogr. D 54: 905-921

Brünger, (1992) X-PLOR, Version 3.1. A System for X-ray Crystallography and NMR, Yale University Press, New Haven, Conn.

Brzezinski et al., (1997) Drug Metab. Dispos. 25: 1089-1096

Case et al., (1997), AMBER 5, University of California, San Francisco

Chabot, (1997) Clin. Pharmacokinet. 33: 245-259

Chemnitius, (1927) J. Prakt. Chem. 117:142

Chen et al., (1998) Biochem. 37: 5101-5117

Cohen et al., (1990) J. Med. Chem. 33: 883-94

Creighton, (1983) Proteins: Structures and Molecular Principles, W. H. Freeman & Co., New York

Danks et al., (1998) Cancer Res. 58: 20-22

Danks et al., (1999) Clin. Cancer Res. 5: 917-924

Ducruix & Geige, (1992) Crystallization of Nucleic Acids and Proteins: A Practical Approach, IRL Press, Oxford, England

Eisen et al., (1994). Proteins 19: 199-221

Esnouf, (1999) Acta. Crystallogr. D 55: 938-940

Gilson et al., (1994) Science 263: 1276-1278

Goodford, (1985) J. Med. Chem. 28: 849-57

Goodsell & Olsen, (1990) Proteins 8: 195-202

Gribskov et al., (1986) Nucl. Acids. Res. 14: 6745

Harel et al., (1993) Proc. Natl. Acad. Sci. U.S.A. 90: 9031-9035

Hauptman, (1997) Curr. Opin. Struct. Biol. 7: 672-80

Helenius & Aebi, (2001) Science 291, 2364-2369

Holm & Sander, (1997) Nucleic Acids Res. 25: 231-234

Joly & Brown, (1986) Toxicol. Appl. Pharmacol. 84: 523-532

Jones et al., (1991) Acta Crystallogr. A 47: 110-119

Kamendulis et al., (1996) J. Pharmacol. Exp. Ther. 279: 713-717

Kaneda et al., (1990) Cancer Res. 50:1715

Khana et al., (2000) Cancer Res. 60: 4725-4728

Kraulis, (1991) Appl. Crystallogr. 24: 946-950

Kroetz et al., (1993) Biochem. 32 (43): 11606-11617

Kryger & Silman, (1999) Structure Fold Des. 7: 297-307

Kryger et al., (1999) Structure Fold Des. 7: 297-307

Kuntz et al., (1992) J. Mol. Biol. 161: 269-88

Kyte & Doolittle, (1982), J. Mol. Biol. 157: 105-132

Lambert, (1997) in Practical Application of Computer-Aided Drug Design, (Charifson, ed.) Marcel-Dekker, New York, pp. 243-303

Laskowski et al., (1993) J. Appl. Crystallogr. 26: 283-291

Lattman, (1985) Method Enzymol., 115: 55-77

Leslie, (1992), Joint CCP4+ESF-EAMCB Newsletter on Protein Crystallography, No. 26

Lotti et al., (1983) Biochem. Pharmacol. 32: 3735-3738

Martin, (1992) J. Med. Chem. 35: 2145-54

Matthews, (1968) J. Mol. Biol. 33: 491-497

McPherson, (1982) Preparation and Analysis of Protein Crystals, John Wiley, New York

McPherson, (1990) Eur. J. Biochem. 189:1-23

McRee, (1992) J. Mol. Graphics 10: 44-47

Meck et al., (2001) Cancer Res. 61: 5083-5089

Merritt & Bacon, (1997) Methods Enzymol. 277: 505-524

Miranker & Karplus, (1991) Proteins 11: 29-34

Mori et al., (1999) FEBS Lett. 458 (1): 17-22

Morton & Potter, (2000) Mol. Biotechnol. 16: 193-202

Morton et al., (2000) Cancer Res. 60: 4206-4210

Navaza & Saludjian, (1997) Methods Enzymol. 276A, 581-594

Navia & Murcko, (1992) Curr. Opin. Struc. Biol. 2: 202-10

Needleman et al., (1970) J. Mol. Biol. 48: 443

Nicholls et al., (1991) Proteins 11: 281-96

Nishibata & Itai, (1991) Tetrahedron 47: 8985

Ollis et al., (1992) Protein Eng. 5: 197-211

Otwinowski, (1991), in Isomorphous Replacement and Anomalous Scattering, (Evans & Leslie, eds.), 80-86, Daresbury Laboratory, Daresbury, United Kingdom

Otwinowski. & Minor, (1993) Data Collection and Processing, Daresbury Laboratories, Warrington, Chesire, United Kingdom

Pearlman et al., (1995) Comput. Phys. Commun. 91: 1-41

Pindel et al., (1997) J. Biol. Chem. 272:14769-14775

Potter et al., (1998) Cancer Res. 58: 2646-2651

Potter et al., (1998) Cancer Res. 58: 3627-3632

Rarey et al., (1996) J. Comput. Aid. Mol. Des. 10:41-54

Read, (1986) Acta Crystallogr. A 42: 140-149

Sambrook et al., (1989) Molecular Cloning: A Laboratory Manual, Cold Spring Harbor Laboratory, New York

Sawada et al., (1991) Chem. Pharm. Bull. 39:1446

Schrag & Cygler, (1993) J. Mol. Biol. 230: 575-591

Schwartz et al., (eds.) (1979), Atlas of Protein Sequence and Structure, National Biomedical Research Foundation, pp. 357-358

Sheldrick, (1990) Acta Cryst. A46: 467

Smith et al., (1981) Adv. Appl. Math. 2:482

Sussman et al., (1993) Chem. Biol Interact. 87: 187-197

Rossmann, (ed.) The Molecular Replacement Method, Gordon & Breach, New York (1972)

Thompson et al., (1994) Nucleic Acids Res. 22: 4673-4680

Van Holde, (1971) Physical Biochemistry, Prentice-Hall, New Jersey, pp. 221-39

Wallace et al., (2001) J. Biol. Chem. 276: 33165-33174

Weber, (1991) Adv. Protein Chem. 41:1-36

Weeks et al., (1993) Acta Cryst. D49: 179

Weirdl et al., (2001) Cancer Res. 61: 5078-5082

Wellner, (1971) Anal. Chem. 43: 597

Wetmur & Davidson, (1968) J. Mol. Biol. 31: 349-70

Williams, (1985) Clin. Pharmacokinet. 10: 392-403

U.S. Pat. No. 4,554,101

U.S. Pat. No. 4,604,463

U.S. Pat. No. 5,463,564

U.S. Pat. No. 5,834,228

U.S. Pat. No. 5,872,011

U.S. Pat. No. 6,008,033

WO 00/025134

WO 00/037077

WO 84/03564

TABLE 1
Comparison of rCE with Related Esterases of Known Function

| | | Triacylglycerol | Cholesterol | Brefeldin A | | Cocaine | CE |
|---|---|---|---|---|---|---|---|
| | RCE | hydrolase | esterase | esterase | Lipase | esterase | (bacterial) |
| PDB entry | — | 1THG | 2BCE | 1JKM | 1JFR | 1JU3 | 1AUO |
| Number of residues superimposed | — | 544 | 532 | 358 | 260 | 228 | 218 |
| Sequence identity (%) | — | 37 | 33 | 20 | 16 | 14 | 14 |
| R.M.S. deviation (Å) | — | 1.8 | 2.2 | 3.2 | 2.8 | 3.9 | 3.2 |
| Catalytic Ser | 221 | 217 | 194 | 202 | 131 | 117 | 114 |
| Catalytic His | 467 | 463 | 435 | 338 | 209 | 287 | 199 |
| Catalytic Glu (Asp)¹ | 353 | 320 | 320 | (308) | (177) | (259) | (168) |
| Distance Ser-O to His-N (Å) | 3.0 | 2.7 | 2.7 | 2.9 | 2.7 | 2.8 | 2.7 |
| Distance His-N to Glu(Asp)-01 (Å)¹ | 8.3 | 2.9 | 3.1 | (3.2) | (3.1) | (2.5) | (3.3) |
| Distance His-N to Glu(Asp)-02 (Å)¹ | 7.6 | 4.5 | 4.7 | (2.5) | (2.8) | (3.8) | (2.6) |

¹The number in parentheses refers to Asp.

TABLE 2
Crystallographic Data And Refinement For Rabbit Carboxylesterase In
Complex With 4PP

| Resolution¹ (Å; highest shell) | 20-2.5 | (2.54-2.5) |
|---|---|---|
| Space Group | R32 | |
| Cell Constants (Å) | a = b = 110.23; c = 282.52 | |
| Total Reflections | 234,266 | |
| Unique Reflections | 22,041 | |
| Mean Redundancy | 10.6 | |
| Wilson B-factor (Å²) | 41.1 | |
| R_{sym} (%)^{1,2} | 7.2 | (42.1) |
| Completeness¹ (%) | 99.7 | (99.1) |
| Mean I/σ¹ | 31.7 | (4.5) |
| R_{cryst} (%)³ | 22.8 | |
| R_{free} (%)⁴ | 29.2 | |
| RMSD^{§} Bond Lengths (Å) | 0.0067 | |
| RMSD^{§} Bond Angles (°) | 1.34 | |
| RMSD^{§} Dihedrals (°) | 22.9 | |
| RMSD^{§} Impropers (°) | 0.91 | |
| Number of Atoms⁵ | | |
| Protein | 3,897 | (60.9) |
| Solvent | 388 | (57.5) |
| Carbohydrate | 99 | (89.9) |
| Ligand | 24 | (75.9) |

¹The number in parentheses is for the highest resolution shell.
²R_{sym} = Σ |I − <I >|/ΣI, where I is the observed intensity and <I> is the average intensity of several symmetry-related observation of that reflection.
³R_{cryst} = Σ ∥Fₒ| | − |F_{c}∥/Σ |Fₒ|, where Fₒ and F_{c} are the observed and calculated structure factors, respectively.
⁴R_{free} = Σ ∥Fₒ| − |F_{c}∥/Σ |Fₒ| for 10% of the data not used at any stage of structural refinement.
⁵The number in parentheses is the mean B-factor (Å²).
^{§}RMSD, root mean square deviation.

Lengthy table referenced here
US20030235811A1-20031225-T00006.XML

Lengthy table referenced here
US20030235811A1-20031225-T00007.XML

Lengthy table referenced here
US20030235811A1-20031225-T00008.XML

Lengthy table referenced here
US20030235811A1-20031225-T00009.XML

Lengthy table referenced here
US20030235811A1-20031225-T00010.XML

It will be understood that various details of the invention can be changed without departing from the scope of the invention. Furthermore, the foregoing description is for the purpose of illustration only, and not for the purpose of limitation.

LENGTHY TABLE(s)

The patent application contains a lengthy table(s) section. A copy of the lengthy table(s) is available in electronic form from the USPTO web site
(http://seqdata.uspto.gov/squence.html?DocID=20030235811). An electronic copy of the lengthy table(s) will also be available from the USPTO
upon request and payment of the fee set forth in 37 CFR 1.19(b)(3).

What is claimed is:

1. A substantially pure mammalian CE polypeptide in crystalline form.

2. The polypeptide of claim 1, wherein the CE is a rabbit CE.

3. The polypeptide of claim 2, wherein the crystalline form is has lattice constants of a=110.23 Å, b=110.23 Å, c=282.52 Å, α=90°, β=90°, γ=120°.

4. The polypeptide of claim 2, wherein the crystalline form is a rhombohedral crystalline form.

5. The polypeptide of claim 2, wherein the crystalline form has a space group of R32.

6. The polypeptide of claim 2, wherein the CE has the amino acid sequence shown in one of SEQ ID NO: 2.

7. The polypeptide of claim 2, wherein the CE is in complex with a ligand.

8. The polypeptide of claim 7, wherein the ligand is 4-piperidino-piperidine.

9. The polypeptide of claim 2, wherein the CE has a crystalline structure further characterized by the coordinates corresponding to Table 3.

10. The polypeptide of claim 2, wherein the crystalline form contains one CE polypeptide in the asymmetric unit.

11. The polypeptide of claim 2, wherein the crystalline form is such that the three-dimensional structure of the crystallized CE polypeptide can be determined to a resolution of about 2.5 Å or better.

12. The polypeptide of claim 2, wherein the crystalline form contains one or more atoms having an atomic weight of 40 grams/mol or greater.

13. The polypeptide of claim 1, wherein the CE is a human CE.

14. The polypeptide of claim 13, wherein the crystalline form is has lattice constants of selected from the group consisting of a=90.0 Å, b=117.0 Å, c=176.0 Å, α=90°, β=95.7°, γ=90°; and a=55.4 Å, b=178.8 Å, c=199.6 Å, α=90°, β=90.2°, γ=90°.

15. The polypeptide of claim 13, wherein the crystalline form is a monoclinic crystalline form.

16. The polypeptide of claim 13, wherein the crystalline form has a space group of P2₁.

17. The polypeptide of claim 13, wherein the CE has the amino acid sequence shown in one of SEQ ID NO: 4.

18. The polypeptide of claim 1, wherein the CE is in complex with a ligand.

19. The polypeptide of claim 18, wherein the ligand is homatropine.

20. The polypeptide of claim 13, wherein the CE has a crystalline structure further characterized by the coordinates corresponding to Table 6.

21. The polypeptide of claim 13, wherein the crystalline form contains six CE polypeptides in the asymmetric unit.

22. The polypeptide of claim 13, wherein the crystalline form is such that the three-dimensional structure of the crystallized CE polypeptide can be determined to a resolution of about 2.8 Å or better.

23. The polypeptide of claim 13, wherein the crystalline form contains one or more atoms having an atomic weight of 40 grams/mol or greater.

24. The polypeptide of claim 18, wherein the ligand is tacrine.

25. The polypeptide of claim 6, wherein the CE has a crystalline structure further characterized by the coordinates corresponding to Table 7.

26. The polypeptide of claim 18, wherein the crystalline form contains six CE polypeptides in the asymmetric unit.

27. The polypeptide of claim 18, wherein the crystalline form is such that the three-dimensional structure of the crystallized CE polypeptide can be determined to a resolution of about 2.4 Å or better.

28. The polypeptide of claim 18, wherein the crystalline form contains one or more atoms having an atomic weight of 40 grams/mol or greater.

29. A method for determining the three-dimensional structure of a crystallized mammalian CE polypeptide to a resolution of about 2.8 Å or better, the method comprising:
(a) crystallizing a mammalian CE polypeptide; and
(b) analyzing the mammalian CE polypeptide to determine the three-dimensional structure of the crystallized mammalian CE polypeptide, whereby the three-dimensional structure of a crystallized mammalian CE ligand binding domain polypeptide is determined to a resolution of about 2.8 Å or better.

30. The method of claim 29, wherein the mammalian CE is rabbit CE.

31. The method of claim 29, wherein the mammalian CE is human CE.

32. The method of claim 29, wherein the analyzing is by X-ray diffraction.

33. The method of claim 29, wherein the crystallization is accomplished by the sitting drop vapor diffusion method, and wherein the mammalian CE polypeptide is mixed with an equal volume of reservoir.

34. The method of claim 33, wherein the reservoir comprises 8% PEG-3350, 0.4 M Li₂SO₄, 0.1M LiCl, 0.1 M NaCl, 0.1 M citrate pH 5.5, 5% glycerol.

35. The method of claim 33, wherein the reservoir comprises 10% (w/v) PEG 3350, 0.1M Li₂SO₄, 0.1M citrate, pH 5.5, and 5% (v/v) glycerol.

36. A method of generating a crystallized mammalian CE ligand binding domain polypeptide, the method comprising:
(a) incubating a solution comprising a mammalian CE ligand binding domain with an equal volume of reservoir; and
(b) crystallizing the mammalian CE polypeptide using the sitting drop method, whereby a crystallized mammalian CE polypeptide is generated.

37. The method of claim 36, wherein the mammalian CE is rabbit CE.

38. The method of claim 37, wherein the rabbit CE comprises SEQ ID NO: 2.

39. The method of claim 36, wherein the mammalian CE is human CE.

40. The method of claim 36, wherein the human CE comprises SEQ ID NO: 4.

41. A crystallized CE polypeptide produced by the method of claim 36.

42. A method of designing a modulator of a CE polypeptide, the method comprising:
(a) designing a potential modulator of a CE polypeptide that will form bonds with amino acids in a ligand binding site based upon a crystalline structure of a CE polypeptide;
(b) synthesizing the modulator; and
(c) determining whether the potential modulator modulates the activity of the CE polypeptide, whereby a modulator of a CE polypeptide is designed.

43. The method of claim 42, wherein the CE is a mammalian CE.

44. The method of claim 43, wherein the CE is a rabbit CE.

45. The method of claim 43, wherein the CE is a human CE.

46. A method of designing a modulator that selectively modulates the activity of a human CE polypeptide to the exclusion of other CE polypeptides, the method comprising:
(a) obtaining a crystalline form of a mammalian CE polypeptide;
(b) evaluating the three-dimensional structure of the crystallized mammalian CE polypeptide; and
(c) synthesizing a potential modulator based on the three-dimensional crystal structure of the crystallized mammalian CE ligand binding domain polypeptide, whereby a modulator that selectively modulates the activity of a human CE polypeptide to the exclusion of other CE polypeptides is designed.

47. The method of claim 46, wherein the mammalian CE is rabbit CE.

48. The method of claim 47, wherein the rabbit CE comprises SEQ ID NO: 2.

49. The method of claim 46, wherein the mammalian CE is human CE.

50. The method of claim 49, wherein the human CE comprises SEQ ID NO: 4.

51. The method of claim 46, wherein the method further comprises contacting a human CE polypeptide with the potential modulator; and assaying the human CE polypeptide for binding of the potential modulator, for a change in activity of the human CE polypeptide, or both.

52. The method of claim 46, wherein the crystalline form is in rhombohedral form.

53. The method of claim 46, wherein the crystalline form is in monoclinic form.

54. The method of claim 46, wherein the crystalline form is such that the three-dimensional structure of the crystallized mammalian CE polypeptide can be determined to a resolution of about 2.8 Å or better.

55. A method for identifying a CE modulator, the method comprising:
(a) providing atomic coordinates of a mammalian CE polypeptide to a computerized modeling system; and
(b) modeling a ligand that fits spatially into a binding cavity or on the surface of the mammalian CE ligand binding domain, whereby a CE modulator is identified.

56. The method of claim 55, wherein the mammalian CE is rabbit CE.

57. The method of claim 56, wherein the rabbit CE comprises SEQ ID NO: 2.

58. The method of claim 55, wherein the mammalian CE is human CE.

59. The method of claim 56, wherein the human CE comprises SEQ ID NO: 4.

60. The method of claim 55, wherein the method further comprises identifying in an assay for CE-mediated activity a modeled ligand that increases or decreases the activity of the CE.

61. A method of identifying a CE modulator that selectively modulates the activity of a CE polypeptide compared to other polypeptides, the method comprising:
(a) providing atomic coordinates of a mammalian CE polypeptide to a computerized modeling system; and
(b) modeling a ligand that fits spatially into a binding cavity or on the surface of a mammalian CE polypeptide and that interacts with conformationally constrained residues of a CE that are conserved among CE orthologs and isoforms, whereby a CE modulator is identified.

62. The method of claim 61, wherein the method further comprises identifying in a biological assay for CE-mediated activity a modeled ligand that selectively binds to the CE polypeptide and increases or decreases the activity of the CE.

63. The method of claim 61, wherein the mammalian CE is rabbit CE.

64. The method of claim 63, wherein the rabbit CE comprises SEQ ID NO: 2.

65. The method of claim 61, wherein the mammalian CE is human CE.

66. The method of claim 65, wherein the rabbit CE comprises SEQ ID NO: 4.

67. A method of designing a modulator of a CE polypeptide, the method comprising:
(a) selecting a candidate CE ligand;
(b) determining which amino acid or amino acids of a CE polypeptide interact with the ligand using a three-dimensional model of a crystallized protein comprising a mammalian CE;
(c) identifying in a biological assay for CE activity a degree to which the ligand modulates the activity of the CE polypeptide;
(d) selecting a chemical modification of the ligand wherein the interaction between the amino acids of the CE polypeptide and the ligand is predicted to be modulated by the chemical modification;
(e) performing the chemical modification on the ligand to form a modified ligand;
(f) contacting the modified ligand with the CE polypeptide;
(g) identifying in a biological assay for CE activity a degree to which the modified ligand modulates the biological activity of the CE polypeptide; and
(h) comparing the biological activity of the CE polypeptide in the presence of modified ligand with the biological activity of the CE polypeptide in the presence of the unmodified ligand, whereby a modulator of a CE polypeptide is designed.

68. The method of claim 67, wherein the mammalian CE polypeptide is a human CE polypeptide.

69. The method of claim 68, wherein the human CE polypeptide comprises SEQ ID NO: 4.

70. The method of claim 68, wherein the human CE is co-crystallized with a ligand.

71. The method of claim 70, wherein the ligand is selected from the group consisting of tacrine and homatropine.

72. The method of claim 67, wherein the mammalian CE is a rabbit CE polypeptide.

73. The method of claim 72, wherein the rabbit CE comprises SEQ ID NO: 2.

74. The method of claim 72, wherein the rabbit CE is co-crystallized with a ligand.

75. The method of claim 74, wherein the ligand is 4-piperidino-piperidine.

76. The method of claim 40, wherein the method further comprises repeating steps (a) through (f), if the biological activity of the CE polypeptide in the presence of the modified ligand varies from the biological activity of the CE polypeptide in the presence of the unmodified ligand.

77. An assay method for identifying a compound that inhibits binding of a ligand to a CE polypeptide, the assay method comprising:
(a) designing a test inhibitor compound capable of modulating CE activity, based on the atomic coordinates of a mammalian CE polypeptide;
(b) synthesizing the test inhibitor compound;
(c) incubating a CE polypeptide with a ligand in the presence of a test inhibitor compound; and
(d) determining an amount of ligand that is bound to the CE polypeptide, wherein decreased binding of ligand to the CE protein in the presence of the test inhibitor compound relative to binding of ligand in the absence of the test inhibitor compound is indicative of inhibition, whereby a compound that inhibits binding of a ligand to a CE polypeptide is identified.

78. The method of claim 77, wherein the mammalian CE is a rabbit CE.

79. The method of claim 78, wherein the rabbit CE comprises SEQ ID NO: 2.

80. The method of claim 78, wherein the rabbit CE is in complex with a ligand.

81. The method of claim 80, wherein the ligand is 4-piperidino-piperidine.

82. The method of claim 77, wherein the mammalian CE is a human CE.

83. The method of claim 82, wherein the human CE comprises SEQ ID NO: 4.

84. The method of claim 82, wherein the human CE is in complex with a ligand.

85. The method of claim 80, wherein the ligand is selected from the group consisting of homatropine and tacrine.

86. A method of modeling a three-dimensional structure of a target CE in complex with a ligand from a template comprising the X-ray structure of a mammalian CE in complex with a ligand, the method comprising:
(a) selecting an X-ray structure of a target CE as a starting model for the target CE;
(b) manipulating the starting model for the target CE as a rigid body to superimpose its backbone atoms onto corresponding backbone atoms of a three-dimensional template structure comprising a mammalian CE in complex with a ligand to form a manipulated model;
(c) making a copy of the ligand from the template structure to form a model of a ligand bound to a template mammalian CE;
(d) merging the model of the ligand into the manipulated model to form a modified model;
(e) removing one or more amino acids from the modified model; and
(f) optimizing side-chain conformations, whereby a three-dimensional structure of a target CE in complex with a ligand is modeled from a template comprising the X-ray structure of a mammalian CE in complex with a ligand.

87. The method of claim 86, wherein the X-ray structure of a target CE is a structure built by homology modeling.

88. The method of claim 86, wherein the mammalian CE is a rabbit CE.

89. The method of claim 88, wherein the rabbit CE comprises the sequence of SEQ ID NO: 2.

90. The method of claim 88, wherein the ligand comprises 4-piperidino-piperidine.

91. The method of claim 88, wherein the three-dimensional template structure is a structure characterized by the coordinates of Table 3.

92. The method of claim 86, wherein the mammalian CE is a human CE.

93. The method of claim 92, wherein the human CE comprises the sequence of SEQ ID NO: 4.

94. The method of claim 92, wherein the ligand is selected from the group consisting of homatropine and tacrine.

95. The method of claim 92, wherein the three-dimensional template structure is a structure characterized by the coordinates of one of Table 6 and Table 7.

96. The method of claim 86, wherein the optimizing comprises varying distance constraints.

97. A method of screening a plurality of compounds for a modulator of a CE polypeptide, the method comprising:
(a) providing a library of test samples;
(b) contacting a crystalline form comprising a mammalian CE in complex with a ligand with each test sample;
(c) detecting an interaction between a test sample and the crystalline mammalian CE polypeptide in complex with a ligand;
(d) identifying a test sample that interacts with the crystalline mammalian CE polypeptide in complex with a ligand; and
(e) isolating a test sample that interacts with the crystalline mammalian CE polypeptide in complex with a ligand, whereby a plurality of compounds is screened for a modulator of a CE ligand binding domain polypeptide.

98. The method of claim 97, wherein the mammalian CE is rabbit CE.

99. The method of claim 98, wherein the rabbit CE comprises SEQ ID NO: 2.

100. The method of claim 99, wherein the ligand is 4-piperidino-piperidine.

101. The method of claim 97, wherein the mammalian CE is human CE.

102. The method of claim 101, wherein the human CE comprises SEQ ID NO: 4.

103. The method of claim 101, wherein the ligand is selected from the group consisting of tacrine and homatropine.

104. The method of claim 97, wherein the test samples are bound to a substrate.

105. The method of claim 104, wherein the test samples are synthesized directly on a substrate.


SEQUENCE LISTING (5 sequences)

SEQ ID NO: 1 - DNA, 1717 bp, Oryctolagus cuniculus
  CDS (11)..(1708)
gaattctgccatgtggctctgtgcattggccctggcctctctcgccgcttgcacggcttgggggcacccgtctgcaccacctgtggtagatactgtgcatggcaaagtcctggggaagttcgtcagcttagaaggatttgcacagcccgtggccgtcttcctgggagtccccttcgccaagccccctcttggatccctgaggtttgcaccaccacagcctgcagaatcatggagccacgtgaagaacaccacctcctaccctcccatgtgctcccaggacgcagtatcagggcatatgctctcggagctcttcaccaacagaaaagagaacatccctcttaagttttctgaagactgcctttacctgaatatttacacccctgctgacctgacaaagagaggcaggctgccggtgatggtgtggatccatggaggtggtctgatggtgggtggagcatcaacctatgatggcctggctctttctgcccatgagaacgtggtggtggtgaccattcagtaccgcctgggcatctggggattcttcagcacaggagatgagcacagccgagggaactggggtcacttggaccaggtggctgcgctgcggtgggtccaggacaacattgccaactttggaggggacccaggctctgtgaccatctttggagagtcagcaggaggtcaaagtgtctctatccttctattatcccccctgaccaagaatctcttccatcgagcaatttccgagagtggcgtggccctcctttccagtctcttcaggaagaacaccaagtccttggctgagaaaattgccatcgaagctgggtgtaaaaccaccacctcggctgtcatggttcactgcctgcgccagaagacagaggaagaactcatggaggtgacattgaaaatgaaatttatggctctagatctagttggcgaccccaaagagaacaccgccttcctgaccactgtgattgatggggtgctgctgccaaaagcacctgcagagattctggcagagaagaaatacaacatgctgccctacatggtgggaatcaaccagcaagagtttggctggattatcccaatgcaaatgctgggctatccactctctgaaggcaaactggaccagaagacagctacagaactcttgtggaagtcctaccccattgtcaatgtctctaaggagctgactccagtggccactgagaagtatttaggagggacagatgaccctgtcaaaaagaaagacttgttcctggacatgcttgcagatttgttatttggtgtcccatctgtgaatgtggctcgtcaccacagagatgctggagcccccacctatatgtatgagtatcggtatcgcccaagcttctcatcagacatgagacccaagacagtgataggggaccatggagatgagatcttctctgtcttaggagccccgtttttaaaagagggtgccacagaagaggagatcaaactgagcaagatggtgatgaaatactgggccaactttgctaggaatgggaatcccaatggagaagggcttcctcaatggccagcatatgactacaaggaaggttacctgcagattggagccaccacccaggcagcccagaaactgaaagacaaggaagtggctttctggactgagctctgggccaaggaggcagcaaggccacgtgagacagagcacattgagctgtgaattgaattc

SEQ ID NO: 2 - protein, 565 aa, Oryctolagus cuniculus
MWLCALALASLAACTAWGHPSAPPVVDTVHGKVLGKFVSLEGFAQPVAVFLGVPFAKPPLGSLRFAPPQPAESWSHVKNTTSYPPMCSQDAVSGHMLSELFTNRKENIPLKFSEDCLYLNIYTPADLTKRGRLPVMVWIHGGGLMVGGASTYDGLALSAHENVVVVTIQYRLGIWGFFSTGDEHSRGNWGHLDQVAALRWVQDNIANFGGDPGSVTIFGESAGGQSVSILLLSPLTKNLFHRAISESGVALLSSLFRKNTKSLAEKIAIEAGCKTTTSAVMVHCLRQKTEEELMEVTLKMKFMALDLVGDPKENTAFLTTVIDGVLLPKAPAEILAEKKYNMLPYMVGINQQEFGWIIPMQMLGYPLSEGKLDQKTATELLWKSYPIVNVSKELTPVATEKYLGGTDDPVKKKDLFLDMLADLLFGVPSVNVARHHRDAGAPTYMYEYRYRPSFSSDMRPKTVIGDHGDEIFSVLGAPFLKEGATEEEIKLSKMVMKYWANFARNGNPNGEGLPQWPAYDYKEGYLQIGATTQAAQKLKDKEVAFWTELWAKEAARPRETEHIEL

SEQ ID NO: 3 - DNA, 1945 bp, Homo sapiens
  CDS (68)..(1750)
tctttcttcatccccgcattcccaatatacccaggccacaagagccgagaactgtcgcccttccacgatgtggctccgtgcctttatcctggccactctctctgcttccgcggcttgggggcatccgtcctcgccacctgtggtggacaccgtgcatggcaaagtgctggggaagttcgtcagcttagaaggatttgcacagcctgtggccattttcctgggaatcccttttggcaagccgcctcttggacccctgaggtttactccaccgcagcctgcagaaccatggagctttgtgaagaatgccacctcgtaccctcctatgtgcacccaagatcccaaggcggggcagttactctcagagctatttacaaaccgaaaggagaacattcctctcaagctttctgaagactgtctttacctcaatatttacactcctgctgacttgaccaagaaaaacaggctgccggtgatggtgtggatccacggaggggggctgatggtgggtgcggcatcaacctatgatgggctggcccttgctgcccatgaaaacgtggtggtggtgaccattcaatatcgcctgggcatctggggattcttcagcacaggggatgaacacagccgggggaactggggtcacctggaccaggtggctgccctgcgctgggtccaggacaacattgccagctttggagggaacccaggctctgtgaccatctttggagagtcagcgggaggagaaagtgtctctgttcttgttttgtctccattggccaagaacctcttccaccgggccatttctgagagtggcgtggccctcacttctgttctggtgaagaaaggtgatgtcaagcccttggctgagcaaattgctatcactgctgggtgcaaaaccaccacctctgctgtcatggttcactgcctgcgacagaagacggaagaggagctcttggagacgacattgaaaatgaaattcttatctctggacttacagggagaccccagagagagtcaaccccttctgggcactgtgattgatgggatgctgctgctgaaaacacctgaagagcttcaagctgaaaggaatttccacactgtcccctacatggtcggaattaacaagcaggagtttggctggttgattccaatgttgatgagctatccactctccgaagggcaactggaccagaagacagccatgtcactcctgtggaagtcctatccccttgtttgcattgctaaggaactgattccagaagccactgagaaatacttaggaggaacagacgacactgtcaaaaagaaagacctgttcctggacttgatagcagatgtgatgtttggtgtcccatctgtgattgtggcccggaaccacagagatgctggagcacccacctacatgtatgagtttcagtaccgtccaagcttctcatcagacatgaaacccaagacggtgataggagaccacggggatgagctcttctccgtctttggggccccatttttaaaagagggtgcctcagaagaggagatcagacttagcaagatggtgatgaaattctgggccaactttgctcgcaatggaaaccccaatggggaagggctgccccactggccagagtacaaccagaaggaagggtatctgcagattggtgccaacacccaggcggcccagaagctgaaggacaaagaagtagctttctggaccaacctctttgccaagaaggcagtggagaagccaccccagacagaacacatagagctgtgaatgaagatccagccggccttgggagcctggaggagcaaagactggggtcttttgcgaaagggattgcaggttcagaaggcatcttaccatggctggggaattgtctggtggtggggggcaggggacagaggccatgaaggagcaagttttgtatttgtgacctcagctttgggaata

SEQ ID NO: 4 - protein, 561 aa, Homo sapiens
MWLRAFILATLSASAAWGHPSSPPVVDTVHGKVLGKFVSLEGFAQPVAIFLGIPFGKPPLGPLRFTPPQPAEPWSFVKNATSYPPMCTQDPKAGQLLSELFTNRKENIPLKLSEDCLYLNIYTPADLTKKNRLPVMVWIHGGGLMVGAASTYDGLALAAHENVVVVTIQYRLGIWGFFSTGDEHSRGNWGHLDQVAALRWVQDNIASFGGNPGSVTIFGESAGGESVSVLVLSPLAKNLFHRAISESGVALTSVLVKKGDVKPLAEQIAITAGCKTTTSAVMVHCLRQKTEEELLETTLKMKFLSLDLQGDPRESQPLLGTVIDGMLLLKTPEELQAERNFHTVPYMVGINKQEFGWLIPMLMSYPLSEGQLDQKTAMSLLWKSYPLVCIAKELIPEATEKYLGGTDDTVKKKDLFLDLIADVMFGVPSVIVARNHRDAGAPTYMYEFQYRPSFSSDMKPKTVIGDHGDELFSVFGAPFLKEGASEEEIRLSKMVMKFWANFARNGNPNGEGLPHWPEYNQKEGYLQIGANTQAAQKLKDKEVAFWTNLFAKKAVEKPPQT

SEQ ID NO: 5 - protein, 550 aa, Homo sapiens
MSAVACGLLLLLVRGQGQDSASPIRTTHTGQVLGSLVHVKGANAGVQTFLGIPFAKPPLGPLRFAPPEPPESWSGVRDGTTHPAMCLQDLTAVESEFLSQFNMTFPSDSMSEDCLYLSIYTPAHSHEGSNLPVMVWIHGGALVFGMASLYDGSMLAALENVVVVIIQYRLGVLGFFSTGDKHATGNWGYLDQVAALRWVQQNIAHFGGNPDRVTIFGESAGGTSVSSLVVSPISQGLFHGAIMESGVALLPGLIASSADVISTVVANLSACDQVDSEALVGCLRGKSKEEILAINKPFKMIPGVVDGVFLPRHPQELLASADFQPVPSIVGVNNNEFGWLIPKVMRIYDTQKEMDREASQAALQKMLTLLMLPPTFGDLLREEYIGDNGDPQTLQAQFQEMMADSMFVIPALQVAHFQCSRAPVYFYEFQHQPSWLKNIRPPHMKADHGDELPFVFRSFFGGNYIKFTEEEEQLSRKMMKYWANFARNGNPNGEGLPHWPLFDQEEQYLQLNLQPAVGRALKAHRLQFWKKALPQKIQELEEPEERHTEL